(19) United States
(12) Patent Application Publication
(10) Pub. No.: US 2015/0031996 A1
(43) Pub. Date: Jan. 29, 2015

(54) SEMICONDUCTING POLYMER NANOPARTICLES AS PHOTOACOUSTIC MOLECULAR IMAGING PROBES

(71) Applicant: The Board of Trustees of the Leland Stanford Junior University, Palo Alto, CA (US)

(72) Inventors: Jianghong Rao, Sunnyvale, CA (US); Kanyi Pu, Mountain View, CA (US); Adam Shuhendler, Stanford, CA (US)

(21) Appl. No.: 14/338,371

(22) Filed: Jul. 23, 2014

Related U.S. Application Data
(60) Provisional application No. 61/857,791, filed on Jul. 24, 2013.

(51) Int. Cl. A61K 49/22; A61B 5/00; A61K 49/00

(52) U.S. Cl. CPC A61K 49/222; A61K 49/0021; A61K 49/0093; A61B 5/0095; A61B 5/0071
USPC 600/431; 424/9.6

(57) ABSTRACT

Provided are nanoparticles comprising an organic photovoltaic semiconductor polymer and a phospholipid, the semiconductor polymer being near-infra red absorbing and generating a detectable photoacoustic signal and a fluorescent emission when irradiated by an incident activation energy. Also provided are methods of molecular imaging, comprising delivering to a subject a plurality of said nanoparticles, irradiating the subject with a first incident energy to generate a first photoacoustic signal, irradiating the subject with a second incident energy to generate a second photoacoustic signal, determining a ratio of the intensities of the two photoacoustic signals, comparing this first ratio with a second ratio determined from the nanoparticles before delivery to the subject, whereby a difference in said first and second ratios indicates ROS degradation of the nanoparticles in the subject; and generating a ratiometric image indicating the difference in said first and second ratios relative to an image of the subject.

### CROSS-REFERENCE TO RELATED APPLICATIONS

This application claims priority to U.S. Provisional Patent Application Ser. No. 61/857,791 entitled “SEMICONDUCTING POLYMER NANOPARTICLES AS PHOTOACOUSTIC MOLECULAR IMAGING PROBES” and filed Jul. 24, 2013, the entirety of which is hereby incorporated by reference.

### STATEMENT REGARDING FEDERALLY SPONSORED RESEARCH OR DEVELOPMENT

This invention was made with government support under NIH National Cancer Institute (NCI) grants R01CA135294 and R21CA138353A2. The government has certain rights in the invention.

### FIELD OF THE DISCLOSURE

The present disclosure relates to photoacoustic semiconducting polymer imaging probes sensitive to reactive oxygen species. The present disclosure further relates to methods for in vivo imaging capable of the detection of oxidative stress using the probes of the disclosure.

### BACKGROUND

Semiconducting Tr-conjugated polymers (SPs) are optically and electronically active materials with many applications ranging from electronic devices (Mei et al., (2013) J. Am. Chem. Soc. 135: 6724-6746; Peet et al., (2007) Nat. Mater. 6: 497-500) and sensors (Sokolov et al., (2012) Accounts Chem. Res. 45: 361-371; Rose et al., (2005) Nature 434: 876-879), to tissue engineering (Sanghvi et al., (2005) Nature Mater. 4: 496-502). Recently, SPs originally designed for organic light-emitting diodes (OLEDs) have been transformed into nanoparticles, representing a new class of photostable fluorescent nanomaterials (Wu et al., (2013) Angew. Chem. Int. Ed. Engl. 52: 3086-3109). Not only are these completely organic materials bright, but they also circumvent the issue of heavy metal ion-induced toxicity to living organisms (Wu et al., (2011) Angew. Chem. Int. Ed. Engl. 50: 3430-3434). These attractive features have generated intense interest in developing SP-based probes for in vivo fluorescence (FL) imaging (Wu et al., (2013) Angew. Chem. Int. Ed. Engl. 52: 3086-3109; Wu et al., (2011) Angew. Chem. Int. Ed. Engl. 50: 3430-3434; Xiong et al., (2012) Nat. Commun. 3: 1193), but they suffer from the general limitations of whole-body optical imaging techniques such as poor spatial resolution and shallow tissue penetration.

Photoacoustic imaging breaks through the optical diffusion limit by integrating optical excitation with ultrasonic detection based on the photoacoustic effect, providing deeper tissue imaging penetration and higher spatial resolution than most whole-body optical imaging techniques (Wang et al., (2012) Science 335: 1458-1462; Ntziachristos et al., (2010) Chem. Rev. 110: 2783-2794). While endogenous molecules (i.e. hemoglobin, melanin) can generate photoacoustic contrast, many physiological and pathological processes often elicit little variation in these intrinsic photoacoustic signals, and thus it is essential to develop exogenous contrast agents for photoacoustic imaging in living subjects (Kim et al., (2010) Chem. Rev. 110: 2756-2782).

Extensive effort has led to the discovery of many materials including small-molecule dyes, metallic nanoparticles and carbon nanotubes as effective photoacoustic contrast agents (Eghtedari et al., (2007) Nano Letters 7: 1914-1918; De la Zerda et al., (2008) Nature Nanotech. 3: 557-562; Kim et al., (2009) Nature Nanotech. 4: 688-694; Lovell et al., (2011) Nature Mater. 10: 324-332; Akers et al., (2011) ACS Nano 5: 173-182; Xia et al., (2011) Acc. Chem. Res. 44: 914-924). Most of these photoacoustic contrast agents share a working mechanism that relies on the simple accumulation through passive (such as the enhanced permeability and retention (EPR) effect) or active (binding to a receptor on the cell surface) targeting at sites of interest to produce an enhanced signal (Wang et al., (2012) Science 335: 1458-1462; Kim et al., (2010) Chem. Rev. 110: 2756-2782; De la Zerda et al., (2008) Nature Nanotech. 3: 557-562; Kim et al., (2009) Nature Nanotech. 4: 688-694; Lovell et al., (2011) Nature Mater. 10: 324-332; Akers et al., (2011) ACS Nano 5: 173-182; Jokerst et al., (2012) ACS Nano 6: 10366-10377). In contrast to this mechanism, activatable molecular imaging probes can undergo an intrinsic signal evolution upon detecting molecular targets or events, and thus provide a real-time correlation between probe state (i.e. activated vs. non-activated) and pathological processes on a molecular level (Lovell et al., (2010) Chem. Rev. 10: 2839-2857; Razgulin et al., (2011) Chem. Soc. Rev. 40: 4186-4216). Activatable probes have been developed for other imaging modalities such as fluorescence (FL) imaging, and have been widely used in biology and medicine (Razgulin et al., (2011) Chem. Soc. Rev. 40: 4186-4216; Weissleder et al., (2003) Nat. Med. 9: 123-128; Melancon et al., (2011) Acc. Chem. Res. 44: 947-956. However, very few NIR activatable probes have been reported for in vivo photoacoustic molecular imaging except for a very recent example demonstrating activatable photoacoustic imaging of matrix metalloproteinase 2/9 activity in FTC133 thyroid tumor xenografts (Levi et al., (2010) J. Am. Chem. Soc. 132: 11264-11269; Levi et al., (2013) Clin. Cancer Res. 19: 1494-1502). Thereby, full utilization of the potential of photoacoustic imaging at a depth and spatial resolution that is unattainable by FL imaging urgently demands new materials amenable to the construction of activatable photoacoustic probes.

As SPs typically used for solar cells were designed to have higher absorption in the NIR region relative to those for OLEDs, these photovoltaic NIR-absorbing SPs were evaluated as building blocks for the development of photoacoustic molecular imaging probes, and found that dependent on the molecular structure of SPs, semiconducting polymer nanoprobes can show better photostability and generate higher photoacoustic signal output than single-wall carbon nanotubes (SWNTs) and gold nanorods (GNRs), two of the best current exogenous photoacoustic contrast nanoagents. The high photoacoustic brightness in the NIR region and favorable small nanoparticle size allowed for photoacoustic lymph node mapping in living mice after a single intravenous administration at a low dosing level (50 μg). By virtue of their facile yet efficient preparation method, an SPN-based dual-peak ratiometric photoacoustic probe was synthesized for both in vitro and in vivo imaging of reactive oxygen species (ROS), which are key integral chemical mediators playing vital roles in the onset and progression of many diseases, ranging from cancer to neurodegenerative and cardiovascular diseases Medzhitov et al., (2008) Nature 454: 428-435; Szabo et al., (2007) Nat. Rev. Drug Discov. 6: 662-680). Accordingly, the disclosure thus provides embodiments of NIR activatable photoacoustic probes capable of reporting the progression of pathological processes in real time, and demonstrates the great potential of SPNs for advanced photoacoustic imaging in biomedical research.

### SUMMARY

One aspect of the disclosure encompasses embodiments of a nanoparticle comprising an organic photovoltaic semiconductor polymer and a phospholipid, where the semiconductor polymer is characterized as near-infra red absorbing and generating a detectable photoacoustic signal and a fluorescent emission when irradiated by an incident activation energy.

Another aspect of the disclosure encompasses embodiments of a pharmaceutically acceptable composition comprising a nanoparticle comprising an organic photovoltaic semiconductor polymer and a phospholipid, wherein the semiconductor polymer is characterized as near-infra red absorbing and generating a detectable photoacoustic signal and a fluorescent emission when irradiated by an incident activation energy.

Yet another aspect of the disclosure encompasses embodiments of a method of molecular imaging, comprising the steps of: (a) delivering to a human or non-human subject a pharmaceutically acceptable composition comprising a plurality of nanoparticles comprising an organic photovoltaic semiconductor polymer, a phospholipid, and a reactive oxygen species (ROS)-inactivated fluorophore, wherein the semiconductor polymer is characterized as near-infra red absorbing and generating a first detectable photoacoustic signal spectrum when irradiated by an incident activation energy; (b) irradiating the human or non-human subject with a first incident energy having a first wavelength and generating a first photoacoustic signal; (c) irradiating the human or non-human subject with a second incident energy having a second wavelength and generating a second photoacoustic signal; (d) determining the intensities of the first and the second photoacoustic signals; (e) determining a first ratio of the intensities of the first and the second photoacoustic signals; (f) comparing said first ratio with a second ratio determined from the nanoparticles before delivery to the human or non-human subject, whereby a difference in said first and second ratios indicates ROS degradation of the nanoparticles in the subject; and (g) generating a ratiometric image indicating the difference in said first and second ratios relative to an image of the subject.

In some embodiments of this aspect of the disclosure, the semiconductor polymer can be poly(cyclopentadithiophene-alt-benzothiadiazole) (PCPDTBT), poly(acenaphthothienopyrazine-alt-benzodithiophene) (PATPBDT), poly[4,6-(dodecyl-thieno[3,4-b]thiophene-2-carboxylate)-alt-2,6-(4,8-dioctoxylbenzo[1,2-b:4,5-b]dit, poly[N-90-heptadecanyl-2,7carbazole-alt-3,6-bis(thiophen-5-yl)-2,5-dioctyl-2,5-dihydropyrrolo[3,4]pyrrole-1,4-dione], poly{4,8-bis[(2-ethylhexyl)oxy]benzo[1,2-b:4,5-b′]dithiophene-2,6-diyl-alt-3-fluoro-2-[(2-ethylhexyl], poly[2,6(4,4′bis(ethylhexyl)dithieno[3,2-b:2′,3′-d]siloleyalt-(1,3-(5-octyl-4H-thieno[3,4-c]pyrrole], or their derivatives.

In some embodiments of this aspect of the disclosure, the phospholipid can be 1,2-dipalmitoyl-sn-glycero-3-phosphocholine (DPPC), 1,2-dinonadecanoyl-sn-glycero-3-phosphocholine, 1,2-diarachidoyl-sn-glycero-3-phosphocholine, 1,2-dibehenoyl-sn-glycero-3-phosphocholine, 1,2-dilignoceroyl-sn-glycero-3-phosphocholine, 1,2-distearoyl-sn-glycero-3-phosphoethanolamine-N-[methoxy(polyethylene glycol)-2000] (ammonium salt), 1,2-dioleoyl-sn-glycero-3-phosphoethanolamine-N-[methoxy(polyethylene glycol)-2000] (ammonium salt), 1,2-dimyristoyl-sn-glycero-3-phosphoethanolamine-N-[methoxy(polyethylene glycol)-350] (ammonium salt), 1,2-distearoyl-sn-glycero-3-phosphoethanolamine-N-[methoxy(polyethylene glycol)-550] (ammonium salt), 1,2-distearoyl-sn-glycero-3-phosphoethanolamine-N-[methoxy(polyethylene glycol)-3000] (ammonium salt), or 1,2-distearoyl-sn-glycero-3-phosphoethanolamine-N-[methoxy(polyethylene glycol)-5000] (ammonium salt).

In some embodiments of this aspect of the disclosure, the fluorophore can be 2-[4′-(β-carboxyethylthio)-7′-(1″,3″,3″-trimethylindolenine)-3′,5′-trimethyleneheptatrien-1-yl]-1,3,3-trimethylindolenium perchlorate (IR775S).

### BRIEF DESCRIPTION OF THE DRAWINGS

Many aspects of the disclosure can be better understood with reference to the following drawings.

FIG. 1 illustrates the molecular structure of PCPDTBT used for the preparation of SPN1.

FIG. 2 illustrates the molecular structure of PATPBDT used for the preparation of SPN2.

FIG. 3 illustrates the molecular structure of the diketopyrrolopyrrole-based SPN3 (PDPPF).

FIG. 4 illustrates the molecular structure of the diketopyrrolopyrrole-based SPN4 (Poly[{2,5-bis(2-hexyldecyl)-2,3,5,6-tetrahydro-3,6-dioxopyrrolo[3,4-c]pyrrole-1,4-diyl}-alt-(4,9-dihydro-4,4,9,9-tetra(4-hexylbenzyl)-sindaceno[1,2-b:5,6-b′ ]-dithiophene-2,7-diyl)} (PDPPDTD).

FIG. 5 illustrates the molecular structure of the diketopyrrolopyrrole-based SPN5 (Poly[{2,5-bis(2-hexyldecyl)-2,3,5,6-tetrahydro-3,6-dioxopyrrolo[3,4-c]pyrrole-1,4-diyl}-alt-thiophene}] (PDPPT).

FIG. 6 illustrates a schematic of the preparation of SPNs through nanoprecipitation. SP can be characterized as the polymer linked by numerous chromophores illustrated as oval beads. DPPC contains a short hydrophobic tail and a charged head and is illustrated as a string with a ball at its end.

FIG. 7 illustrates photographic images of the SPN solutions (10 μg/mL).

FIG. 8 illustrates representative TEM images of SPN1 (left) and SPN2 (right).

FIG. 9 illustrates representative DLS profiles of SPNs.

FIG. 10 illustrates UV-Vis absorption spectra of SPNs.

FIG. 11 illustrates photoacoustic spectra of SPNs.

FIG. 12 is a graph illustrating photoacoustic amplitudes of SPNs at 700 nm in an agar phantom as a function of mass concentration. R²=0.998 and 0.997 for SPN1 and SPN2, respectively.

FIG. 13 illustrates UV-Vis absorption spectra of SPN3, SPN4, and SPN5.

FIG. 14 illustrates photoacoustic spectra of SPN3, SPN4, and SPN5.

FIG. 15A illustrates photoacoustic amplitudes of the nanoparticles based on the same mass (25 μg/mL) (top) and molar (48 nM) (bottom) concentrations in an agar phantom.

FIG. 15B illustrates changes in PA signals of these nanoparticles in agar phantoms under continuous laser scanning for 20 min.

FIG. 15C illustrates photoacoustic amplitudes of the nanoparticle-matrigel inclusions (30 μL) in the subcutaneous dorsal space of living mice as a function of nanoparticle mass concentration. The dashed line shows the tissue background signal calculated as the average PA signal in areas where no nanoparticles were injected. R²=0.995, 0.991 and 0.993 for SPN1, SWNT, and GNR, respectively.

FIG. 15D illustrates photoacoustic/US co-registered images of the nanoparticle-matrigel inclusions at a concentration of 8 μg/mL. The images represent transverse slices through the subcutaneous inclusions (dotted circles). A single laser pulse at 700 nm with a laser fluence of 9 mJ cm⁻² and a pulse repetition rate of 20 Hz were used for all experiments. Data represent mean±standard deviation of three measurements.

FIGS. 16A-16D illustrate in vivo and ex vivo photoacoustic and FL imaging of lymph nodes using SPN1.

FIG. 16A illustrates US (top) and photoacoustic/US co-registered (bottom) images of mouse lymph nodes following tail vain injection of SPN1 (50 μg). The images represent transverse slices through the lymph nodes. BLN: brachial lymph node; ILN: inguinal lymph node; SCLN: superficial cervical lymph node.

FIG. 16B illustrates FL/bright-field images of the corresponding mouse.

FIG. 16C illustrates ex vivo photoacoustic/US co-registered (top) and FL/bright-field (bottom) images of resected lymph nodes from the corresponding mouse (left) or a control mouse without SPN1 injection (right) in an agar phantom.

FIG. 16D illustrates ex vivo quantification of photoacoustic and FL signals of the lymph nodes from SPN1-administrated mice (n=4) and control mice (n=4). *Significant difference in both photoacoustic and FL signals between the lymph nodes from SPN1-administrated and control mice (p<0.05).

FIGS. 17A-17E illustrate in vitro characterization of RSPN for ROS sensing.

FIG. 17A illustrates a ROS sensing mechanism.

FIG. 17B illustrates representative photoacoustic spectra of RSPN in the absence and presence of ROS. [RSPN]=5 μg/mL, [ROS]=5 μM.

FIG. 17C illustrates the ratio of photoacoustic amplitude at 700 nm to that at 820 nm (PA₇₀₀/PA₈₂₀) after treatment with indicated ROS (5 μM).

FIG. 17D illustrates photoacoustic images of macrophage RAW264.7 cell pellets (1.5×10⁶ cells) without (top) or with (middle) stimulation with LPS/INF-γ, and with NAC protection (bottom). Cell pellets were inserted into an agar phantom and imaged with pulsed laser tuned to (i) 700 nm or (ii) 820 nm; (iii) overlays of images from columns i and ii. The cells were incubated with RSPN (6 μg/mL) for 3 h before trypsinization.

FIG. 17E illustrates the quantification of the absorption ratio (PA₇₀₀/PA₈₂₀) for macrophage cell pellets with and without LPS/INF-γ or LPS/INF-γ/NAC treatment. The error bars represent the standard deviation from four measurements. *Significant difference in PA₇₀₀/PA₈₂₀ between LPS/INF-γ treated and untreated or NAC-protected cell pellets (p<0.05).

FIGS. 18A and 18B illustrate in vivo photoacoustic imaging of ROS generation from a mouse model of acute edema using RSPN (n=3).

FIG. 18A illustrates photoacoustic /US overlaid images of saline-treated (i) and zymosan-treated (ii) regions in the thigh of living mice (n=3). RSPN (3 μg in 50 μL) was intramuscularly injected into the thigh 20 min after zymosan treatment.

FIG. 18B illustrates the ratio of photoacoustic amplitude at 700 nm to that at 820 nm (PA₇₀₀/PA₈₂₀) as a function of time post-injection of RSPN. *Significant difference in PA₇₀₀/PA₈₂₀ between zymosan-treated and saline-treated mice at all time points starting from 10 min (p<0.05).

FIGS. 19A and 19B are graphs illustrating stability studies of SPN1 (FIG. 13A) and SPN2 (FIG. 6B) in 1×PBS (pH=7.4) over 6 days.

FIG. 20A illustrates a fluorescence spectrum of SPN1 in 1×PBS (pH=7.4) upon excitation at 680 nm.

FIG. 20B illustrates fluorescence images of the SPN1 solutions (pH=7.4) at different concentrations. Images were obtained from IVIS with excitation at 680 nm and emission at 840 nm.

FIG. 21A illustrates a schematic of the tissue-mimicking phantom implanted with two PE tubes filled with SPN1 at a concentration of 40 (Tube 1) and 20 (Tube 2) μg/mL, respectively.

FIG. 21B illustrates a fluorescence/bright-field overlaid top-view image of the tissue-mimicking phantom.

FIG. 21C illustrates a 3D photoacoustic /US co-registered image of the tissue-mimicking phantom.

FIG. 21D illustrates a 2D photoacoustic top-view image of the tissue-mimicking phantom.

FIG. 22A illustrates a fluorescence/bright-field overlaid image of a PE tube filled with SPN1.

FIG. 22B illustrates a fluorescence/bright-field overlaid image of a mouse with the SPN1-filled PE tube implanted subcutaneously at the dorsal aspect of the leg. Dashed line encircles the location of the tube.

FIG. 22C illustrates a photoacoustic (top) and photoacoustic /US co-registered (bottom) images of the SPN1-filled PE tube in an agar phantom. White arrows indicate the sidewalls of tubes. [SPN1]=20 μg/mL.

FIG. 22D illustrates a photoacoustic (top) and photoacoustic /US co-registered (bottom) images of the mouse leg subcutaneously implanted with tubes filled with the nanoparticle solution (left) or water (right), respectively. White arrows indicate the sidewalls of tubes. [SPN1]=20 μg/mL. Only photoacoustic signals at the walls of the tube are visible because only the higher frequency components of the photoacoustic generated pressure wave are detected.

FIG. 23A illustrates photoacoustic /US co-registered images of a representative mouse before tail vain injection of SPN1.

FIG. 23B illustrates photoacoustic /US co-registered FL/bright-field (b) images of a representative mouse before tail vain injection of SPN1.

FIG. 24A illustrates a fluorescence/bright-field image of a mouse administrated SPN1 (50 μg) after necropsy and skin resection to expose the intact peritoneal cavity, lymph nodes, and milk line of the mouse. 1, 2, 6, and 7 are superficial cervical lymph nodes (SCLNs), 3 and 8 are axillary lymph nodes (ALNs), 4 and 9 are brachial lymph nodes (BLNs), and 5 and 10 are inguinal lymph nodes (ILNs).

FIG. 24B illustrates a fluorescence/bright-field image of the organs from the corresponding mouse.

FIG. 24C illustrates the quantification of the fluorescence signals of the organs from mice (n=4) administered SPN1 (50 μg) via tail vein injection.

FIG. 25A-25C illustrate a ROS stability study of SPN1 and GNR.

FIG. 25A illustrates UV spectra of GNR in the absence or presence of ClO⁻ at concentrations ranging from 0 to 12 μM at intervals of 2 μM.

FIG. 25B illustrates UV spectra of SPN1 in the absence or presence of ClO⁻ at concentrations ranging from 0 to 12 μM at intervals of 2 μM.

FIG. 25C is a graph illustrating changes in the absorption of SPN1 at 700 nm after incubation with ROS for 15 min. [SPN1]=[GNR]=5 μg/mL; [ROS]=12 μM. The data show that SPN1 is highly resistant to ROS.

FIGS. 26A and 26B illustrate a kinetic study of ROS-mediated degradation of 1R7755 by detecting its fluorescence decrease at 800 nm after addition of ROS.

FIG. 26A illustrates Fluorescence spectra of 1R775S.

FIG. 26B illustrates a time course of the fluorescence of 1R775S at 800 nm after addition of indicated ROS (FIG. 26B top: ONOO⁻, ClO⁻; FIG. 26B bottom: ¹O₂ and H₂O₂) in 1×PBS (pH=7.4). [1R775]=1 μg/mL. [ROS]=2 μM. Dashed line (control) shows the intensity level of the intact 1R775S. The rapidly decreased fluorescence indicates that 1R775S undergoes nearly instant degradation in the presence of peroxynitrite (ONOO⁻) and hypochlorite (ClO⁻).

FIG. 27A illustrates a representative TEM of RSPN.

FIG. 27B illustrates a size stability study of RSPN in 1×PBS (pH=7.4) over time. Error bar indicates SD.

FIG. 28A illustrates representative UV-Vis spectra of RSPN in the absence and presence of ROS. [RSPN]=5 μg mL⁻ and [ROS]=5 μM.

FIG. 28B illustrates normalized photoacoustic spectra of IR7755 at different concentrations in agar phantom. The mass concentration of 0.10 mg/mL is equal to the molar concentration of 0.20 mM.

FIG. 29A is a graph illustrating the in vitro viability of RAW264.7 cells treated with SPN solutions at concentrations of 5, 10, 20, or 30 μg/mL for 24 h. The percentage cell viability of treated cells was calculated relative to that of untreated cells with control viability defined as 100%. Error bars are standard deviation.

FIG. 29B illustrates fluorescence (left) and fluorescence/bright-field overlay (right) images of live macrophage RAW 264.7 cells after incubation with SPN probe (6 μg mL⁻¹) for 3 h.

FIG. 29C illustrates confocal fluorescence (left) and fluorescence/bright-field overlaid (right) images of fixed macrophage RAW 264.7 cells after incubation with SPN probe (6 μg mL⁻¹) for 3 h. Cell nucleus was stained by DAPI.

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, toxicology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

Prior to describing the various embodiments, the following definitions are provided and should be used unless otherwise indicated.

### DEFINITIONS

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “reactive oxygen and nitrogen species (RONS)” as used herein refers to oxygen- or nitrogen-containing molecules that are capable of producing oxidative damage to other molecules. Many, but not all, RONS are free radicals. A radical is a group of atoms which behaves as a unit and has one or more unpaired electrons. Examples include, but are not limited to: H₂O₂ (hydrogen peroxide), *O₂⁻ (superoxide radical), *OH, (hydroxyl radical), ONOO⁻ (peroxynitrite), O₂¹ (singlet oxygen), O³ (ozone), *NO (nitric oxide), and *NO₂ (nitrogen dioxide).

The term “nanoparticle” as used herein refers to a particle having a diameter of between about 1 and about 1000 nm. Similarly, by the term “nanoparticles” is meant a plurality of particles having an average diameter of between about 1 and about 1000 nm.

The terms “core” or “nanoparticle core” as used herein refers to the inner portion of nanoparticle. A core can substantially include a single homogeneous monoatomic or polyatomic material. A core can be crystalline, polycrystalline, or amorphous. A core may be “defect” free or contain a range of defect densities. In this case, “defect” can refer to any crystal stacking error, vacancy, insertion, or impurity entity (e.g., a dopant) placed within the material forming the core. Impurities can be atomic or molecular.

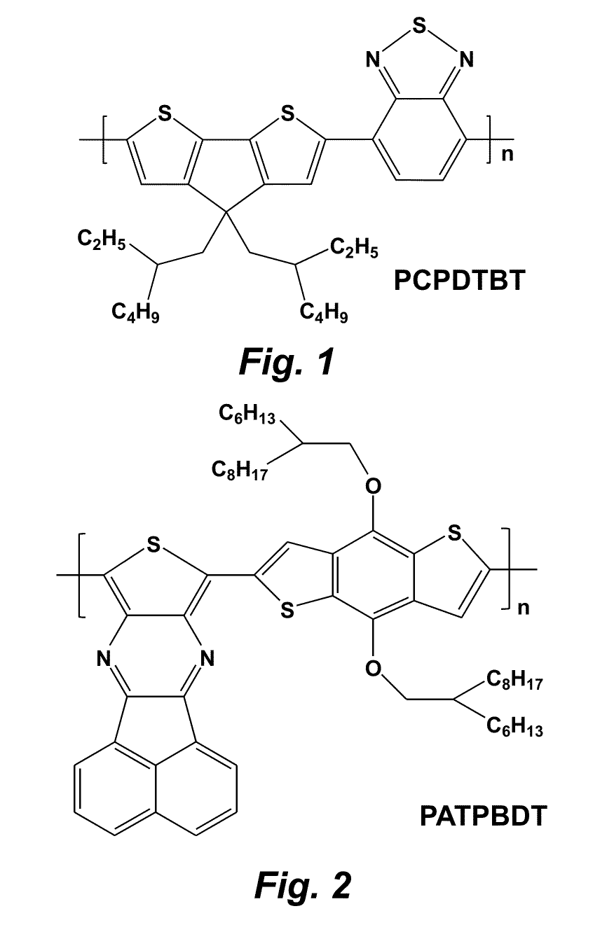

In particular, it is understood that the nanoparticle core of the compositions of the disclosure can comprise a semiconducting polymer that may emit an acoustic signal when irradiated by a suitable incident energy. The semiconducting polymers suitable for use in the compositions of the disclosure may advantageously include, but are not limited to, poly(cyclopentadithiophene-alt-benzothiadiazole) (PCPDTBT) (Muhlbacher et al., (2006) Adv. Mater. 18: 2884-2889) and poly(acenaphthothienopyrazine-alt-benzodithiophene) (PATPBDT) as shown in FIGS. 1 and 2. Other suitable semiconducting polymers include, but are not limited to, such as poly[4,6-(dodecyl-thieno[3,4-b]thiophene-2-carboxylate)-alt-2,6-(4,8-dioctoxylbenzo[1,2-b:4,5-b]dit, poly[N-90-heptadecanyl-2,7carbazole-alt-3,6-bis(thiophen-5-yl)-2,5-dioctyl-2,5-dihydropyrrolo[3,4]pyrrole-1,4-dione], poly{4,8-bis[(2-ethylhexyl)oxy]benzo[1,2-b:4,5-b′]dithiophene-2,6-diyl-alt-3-fluoro-2-[(2-ethylhexyl], poly[2,6(4,4′bis(ethylhexyl)dithieno[3,2-b:2′,3′-d]silole)-alt-(1,3-(5-octyl-4H-thieno[3,4-c]pyrrole], or their derivatives.

Nanoparticles of the disclosure may further comprise a “coat” or “shell” of a second material that surrounds the core. A coat can include a layer of material, either organic or inorganic, that covers or substantially covers the surface of the core of a nanoparticle. A coat may be crystalline, polycrystalline, or amorphous or may comprise, for example, hydrophilic regions of a molecule where hydrophobic regions thereof are either conjugated to or are integral to the underlying nanoparticle core.

A coat or shell may be “complete”, indicating that the coat substantially or completely surrounds the outer surface of the core (e.g., substantially all surface atoms of the core are covered with coat material). Alternatively, the coat may be “incomplete” such that the coat partially surrounds the outer surface of the core (e.g., partial coverage of the surface core atoms is achieved). In addition, it is possible to create coats of a variety of thicknesses, which can be defined in terms of the number of “monolayers” of coat material that are bound to each core. A “monolayer” is a term known in the art referring to a single complete coating of a material (with no additional material added beyond complete coverage). Incomplete monolayers may be either homogeneous or inhomogeneous, forming islands or clumps of coat material on the surface of the nanoparticle core. Coats may be either uniform or non-uniform in thickness. In the case of a coat having non-uniform thickness, it is possible to have an “incomplete coat” that contains more than one monolayer of coat material. A coat may optionally comprise multiple layers of a plurality of materials in an onion-like structure, such that each material acts as a coat for the next-most inner layer. Between each layer there is optionally an interface region. The term “coat” as used herein describes coats formed from substantially one material as well as a plurality of materials that can, for example, be arranged as multi-layer coats.

It will be understood by one of ordinary skill in the art that when referring to a population of nanoparticles as being of a particular “size”, what is meant is that the population is made up of a distribution of sizes around the stated “size”. Unless otherwise stated, the “size” used to describe a particular population of nanoparticles will be the mode of the size distribution (i.e., the peak size). By reference to the “size” of a nanoparticle is meant the length of the largest straight dimension of the nanoparticle. For example, the size of a perfectly spherical nanoparticle is its diameter.

The term “fluorophore” as used herein refers to a component of a molecule that causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore. Fluorophores for use in the compositions of the disclosure include, but are not limited to, fluorescein isothiocyanate (FITC), a reactive derivative of fluorescein, which has been one of the most common fluorophores chemically attached to other, non-fluorescent, molecules to create new fluorescent molecules for a variety of applications. Other historically common fluorophores are derivatives of rhodamine (TRITC), coumarin, and cyanine. Newer generations of fluorophores such as the ALEXA FLUORS® and the DYLIGHT FLUORS® are generally more photostable, brighter, and less pH-sensitive than other standard dyes of comparable excitation and emission.

The term “dye” as used herein refers to any reporter group whose presence can be detected by its light absorbing or light emitting properties. For example, Cy5 is a reactive water-soluble fluorescent dye of the cyanine dye family. Cy5 is fluorescent in the red region (about 650 to about 670 nm). It may be synthesized with reactive groups on either one or both of the nitrogen side chains so that they can be chemically linked to either nucleic acids or protein molecules. Labeling is done for visualization and quantification purposes. Cy5 is excited maximally at about 649 nm and emits maximally at about 670 nm, in the far red part of the spectrum; quantum yield is 0.28. FW=792. Suitable fluorophores(chromes) for the probes of the disclosure may be selected from, but not intended to be limited to, fluorescein isothiocyanate (FITC, green), cyanine dyes Cy2, Cy3, Cy3.5, Cy5, Cy5.5 Cy7, Cy7.5 (ranging from green to near-infrared), Texas Red, and the like. Derivatives of these dyes for use in the embodiments of the disclosure may be, but are not limited to, Cy dyes (Amersham Bioscience), Alexa Fluors (Molecular Probes Inc.,), HILYTE® Fluors (AnaSpec), and DYLITE® Fluors (Pierce, Inc.).

The term “fluorescent acceptor molecule” as used herein refers to any molecule that can accept energy emitted as a result of the activity of a bioluminescent donor protein, and re-emit it as light energy.

The terms “fluorescence quencher” or “quencher” as used herein refer to a molecule that interferes with the fluorescence emitted by a fluorophore or bioluminescent polypeptide. This quencher can be selected from non-fluorescent aromatic molecules, to avoid parasitic emissions. Exemplary quenchers include, but are not limited to, Dabsyl or a BLACK HOLE QUENCH ER® that are non-fluorescent aromatic molecules that prevent the emission of fluorescence when they are physically near a fluorophore. The quencher can also be, but is not limited to, a fluorescent molecule, for example TAMRA (carboxytetramethylrhodamine). A particularly advantageous quencher suitable for use in the compositions of the disclosure is a modified dye such as IR-775-COOH. When the quencher is a fluorescent dye, its fluorescence wavelength is typically substantially different from that of the reporter dye.

The terms “quench” or “quenches” or “quenching” or “quenched” as used herein refer to reducing the signal produced by a molecule. It includes, but is not limited to, reducing the signal produced to zero or to below a detectable limit. Hence, a given molecule can be “quenched” by, for example, another molecule and still produce a detectable signal, albeit the size of the signal produced by the quenched molecule can be smaller when the molecule is quenched than when the molecule is not quenched.

The term “detectable signal emitter” as used herein refers, for the purposes of the specification or claims, to a label molecule that is incorporated indirectly or directly into a nanoparticle, wherein the label molecule facilitates the detection of the nanoparticle in which it is incorporated, for example when the nanoparticle of the disclosure is at a site of inflammation and activated by interaction between the nanoparticle or the quencher component thereof and a RONS. Thus, “detectable signal emitter” is used synonymously with “label molecule”.

The term “NH₂-functionalized conjugated polymer” as used herein refers to a nanoparticle formed by co-condensing one or more types of monomer to form a polymer and wherein on the outer surface of said nanoparticle are located amine groups that are available for conjugating with another molecular entity that may have such as a reactive carboxyl group thereon.

The term “detectable” refers to the ability to detect a signal over the background signal.

The term “detecting” refers to detecting a signal generated by one or more photoacoustic probes. It should be noted that reference to detecting a signal from a photoacoustic probe also includes detecting a signal from a plurality of photoacoustic probes. In some embodiments, a signal may only be detected that is produced by a plurality of photoacoustic probes. Additional details regarding detecting signals (e.g., acoustic signals) are described below.

The term “acoustic detectable signal” is a signal derived from a probe of the present disclosure that absorbs light and converts absorbed energy into thermal energy that causes generation of acoustic signal through a process of thermal expansion. The acoustic detectable signal is detectable and distinguishable from other background acoustic signals that are generated from the host. In other words, there is a measurable and statistically significant difference (e.g., a statistically significant difference is enough of a difference to distinguish among the acoustic detectable signal and the background, such as about 0.1%, 1%, 3%, 5%, 10%, 15%, 20%, 25%, 30%, or 40% or more difference between the acoustic detectable signal and the background) between acoustic detectable signal and the background. Standards and/or calibration curves can be used to determine the relative intensity of the acoustic detectable signal and/or the background.

The detectable signal is defined as an amount sufficient to yield an acceptable image using equipment that is available for pre-clinical use. A detectable signal maybe generated by one or more administrations of the probes of the present disclosure. The amount administered can vary according to factors such as the degree of susceptibility of the individual, the age, sex, and weight of the individual, idiosyncratic responses of the individual, the dosimetry, and the like. The amount administered can also vary according to instrument and digital processing related factors.

The term “in vivo imaging” as used herein refers to methods or processes in which the structural, functional, or physiological state of a living being is examinable without the need for a life-ending sacrifice.

The term “non-invasive in vivo imaging” as used herein refers to methods or processes in which the structural, functional, or physiological state of a being is examinable by remote physical probing without the need for breaching the physical integrity of the outer (skin) or inner (accessible orifices) surfaces of the body.

The term “optical energy” as used herein refers to electromagnetic radiation between the wavelengths of about 350 nm to about 800 nm and which can be absorbed by the dyes or cellulose-based nanoparticles of the embodiments of the photoacoustic probes of the disclosure.

The term “optical energy” may be construed to include laser light energy or non-laser energy.

The term “thermal energy” as used herein refers to electromagnetic radiation of wavelengths between about 700 nm and about 1000 nm and which can increase the temperature of a medium exposed to such radiation.

The term “acoustic signal” as used herein refers to a sound wave produced by one of several processes, methods, interactions, or the like (including light absorption) that provides a signal that can then be detected and quantitated with regard to its frequency and/or amplitude. The acoustic signal can be generated from one or more photoacoustic probes. In some embodiments, the acoustic signal may need to be the sum of each of the individual photoacoustic probes or groups of photoacoustic probes. In some embodiments, the acoustic signal can be generated from a summation, an integration, or other mathematical process, formula, or algorithm, where the acoustic signal is from one or more photoacoustic probes. The summation, the integration, or other mathematical process, formula, or algorithm can be used to generate the acoustic signal so that the acoustic signal can be distinguished from background noise and the like.

The term “photoacoustic imaging” as used herein refers to a biomedical imaging modality based on the photoacoustic effect. The photoacoustic effect refers to when light energy is transformed into kinetic energy of the sample. This results in local heating, and thus a pressure wave or sound. In photoacoustic imaging, non-ionizing laser pulses are delivered into biological tissues. Some of the delivered energy will be absorbed and converted into heat, leading to transient thermoelastic expansion and thus ultrasonic emission at MHz frequencies. The generated ultrasonic waves are detected by ultrasonic transducers to form images. Since optical absorption has been associated with physiological properties, such as hemoglobin concentration or oxygen saturation, the ultrasonic emission (i.e. photoacoustic signal) reveals physiologically specific optical absorption contrast. 2D or 3D images of the targeted areas are possible.

The term “therapeutic agent” as used herein refers to any compound or combination of said compounds that can be advantageously added to the SNPs of the disclosure for delivery to, and modulation of the bioactivity of a cell or population of cells in a recipient subject, said cells also desired to be imaged by the SNPs. For example, but not intended to be limiting, such agents may reduce the proliferative capacity of a cancer cell or tumor, or reduce the manifestation of an inflammation that is imaged by the SNP(s). Advantageous anti-cancer drugs that can be co-administered with SNPs of the present disclosure include, but are not limited to, acivicin; aclarubicin; acodazole hydrochloride; acronine; adriamycin; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interferon α-2a; interferon α-2b; interferon α-n1; interferon α-n3; interferon β-ia; interferon γ-ib; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; taxol; tecogalan sodium; tegafur; teloxantrone hydrochloride; temoporfin; teniposide; teroxirone; testolactone; thiamiprine; thioguanine; thiotepa; tiazofuirin; tirapazamine; topotecan hydrochloride; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Additional antineoplastic agents include those disclosed in Chapter 52, Antineoplastic Agents (Calabresi & Chabner), and the introduction thereto, 1202-1263, of Goodman and Gilman's “The Pharmacological Basis of Therapeutics”, Eighth Edition, 1990, McGraw-Hill, Inc. (Health Professions Division).

Anti-inflammatory drugs that can be administered in combination with SNPs of the present disclosure include, but are not limited to, alclofenac; alclometasone dipropionate; algestone acetonide; alpha amylase; amcinafal; amcinafide; amfenac sodium; amiprilose hydrochloride; anakinra; anirolac; anitrazafen; apazone; balsalazide disodium; bendazac; benoxaprofen; benzydamine hydrochloride; bromelains; broperamole; budesonide; carprofen; cicloprofen; cintazone; cliprofen; clobetasol propionate; clobetasone butyrate; clopirac; cloticasone propionate; cormethasone acetate; cortodoxone; deflazacort; desonide; desoximetasone; dexamethasone dipropionate; diclofenac potassium; diclofenac sodium; diflorasone diacetate; diflumidone sodium; diflunisal; difluprednate; diftalone; dimethyl sulfoxide; drocinonide; endrysone; enlimomab; enolicam sodium; epirizole; etodolac; etofenamate; felbinac; fenamole; fenbufen; fenclofenac; fenclorac; fendosal; fenpipalone; fentiazac; flazalone; fluazacort; flufenamic acid; flumizole; flunisolide acetate; flunixin; flunixin meglumine; fluocortin butyl; fluorometholone acetate; fluquazone; flurbiprofen; fluretofen; fluticasone propionate; furaprofen; furobufen; halcinonide; halobetasol propionate; halopredone acetate; ibufenac; ibuprofen; ibuprofen aluminum; ibuprofen piconol; ilonidap; indomethacin; indomethacin sodium; indoprofen; indoxole; intrazole; isoflupredone acetate; isoxepac; isoxicam; ketoprofen; lofemizole hydrochloride; lomoxicam; loteprednol etabonate; meclofenamate sodium; meclofenamic acid; meclorisone dibutyrate; mefenamic acid; mesalamine; meseclazone; methylprednisolone suleptanate; morniflumate; nabumetone; naproxen; naproxen sodium; naproxol; nimazone; olsalazine sodium; orgotein; orpanoxin; oxaprozin; oxyphenbutazone; paranyline hydrochloride; pentosan polysulfate sodium; phenbutazone sodium glycerate; pirfenidone; piroxicam; piroxicam cinnamate; piroxicam olamine; pirprofen; prednazate; prifelone; prodolic acid; proquazone; proxazole; proxazole citrate; rimexolone; romazarit; salcolex; salnacedin; salsalate; sanguinarium chloride; seclazone; sermetacin; sudoxicam; sulindac; suprofen; talmetacin; talniflumate; talosalate; tebufelone; tenidap; tenidap sodium; tenoxicam; tesicam; tesimide; tetrydamine; tiopinac; tixocortol pivalate; tolmetin; tolmetin sodium; triclonide; triflumidate; zidometacin; zomepirac sodium.

Further definitions are provided in context below. Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein.

### DESCRIPTION

The disclosure provides embodiments of near-infrared (NIR) absorbing semiconducting polymer nanoparticles (SPNs). Such nanoparticles can serve as an efficient and stable nanoplatform to convert photons into ultrasound waves, permitting in vivo photoacoustic (PA) molecular imaging with fine spatial resolution at depths beyond the optical diffusion limit. As SPs typically used for solar cells were designed to have higher absorption in the near-infrared region relative to those for OLEDs, these photovoltaic near-infrared -absorbing SPs were evaluated as building blocks for the development of PA molecular imaging probes, and it was unexpectedly found that dependent on the molecular structure of SPs, SPNs can show better photostability and generate higher PA signal output than single-wall carbon nanotubes (SWNTs) and gold nanorods (GNRs), two of the best current exogenous PA contrast nanoagents. The high PA brightness in the near-infrared region and favorable small nanoparticle size allowed for PA lymph node mapping in living mice after a single intravenous administration at a low dosing level (50 μg).

By virtue of their facile yet efficient preparation method, SPN-based dual-peak ratiometric PA probes were synthesized for both in vitro and in vivo imaging of reactive oxygen species (ROS), which are key integral chemical mediators playing vital roles in the onset and progression of many diseases, ranging from cancer to neurodegenerative and cardiovascular diseases (Medzhitov et al., (2008) Nature 454: 428-435; Szabo et al., (2007) Nat. Rev. Drug Discov. 6: 662-680). Accordingly, the present disclosure provides embodiments of near-infrared activatable PA probes useful in methods for reporting the progression of pathological processes in real time, and demonstrates advantageous use of SPNs for PA imaging in biomedical research.

The disclosure encompasses embodiments of nanoparticle probes that may be detected by their emission of photoacoustic signals when irradiated with at least one incident energy source. It has been found that the nanoparticles of the disclosure, which can combine a semiconducting polymer and a fluorescent dye, have a characteristic first spectrum of photoacoustic emission as a function of the wavelength of the incident energy source. However, in the presence of a reactive oxygen or nitrogen species the fluorescent dye is degraded and the emitted photoacoustic spectrum changes such that the emission at certain incident wavelength(s) is diminished in intensity or eliminated altogether. It has further been found that at certain incident light wavelengths the emitted photoacoustic signal is subject to little if any decrease.

Accordingly, by determining the ratio of the intensity of the variable photoacoustic signal to the intensity of the non-variable photoacoustic signal it is possible to determine the degree of exposure of the nanoparticles to a reactive oxygen or nitrogen species. Advantageously, the emission of a reactive oxygen or nitrogen species (RONS)-insensitive signal can allow the generation of an image for indicating the location of the nanoparticles in a recipient subject. Determining the ratio of the intensities of the photoacoustic signals at the sensitive and insensitive incident wavelengths and identifying where a change in the ratio has occurred allows the generation of an image of identifying the location of RONS in the subject and hence the location of inflammation or other tissue injury or pathologies.

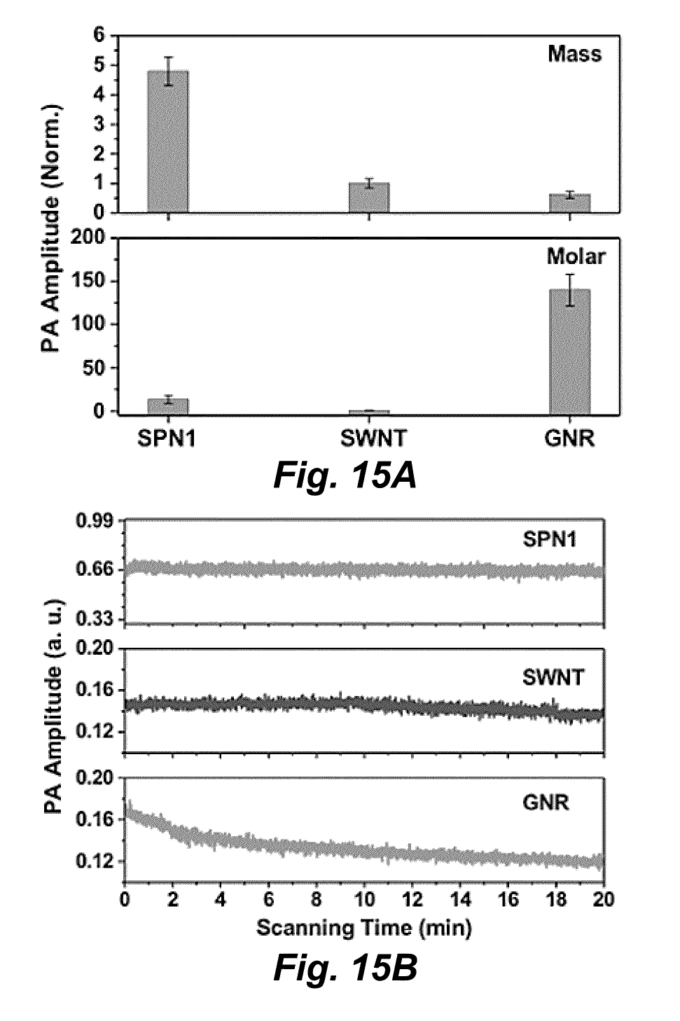

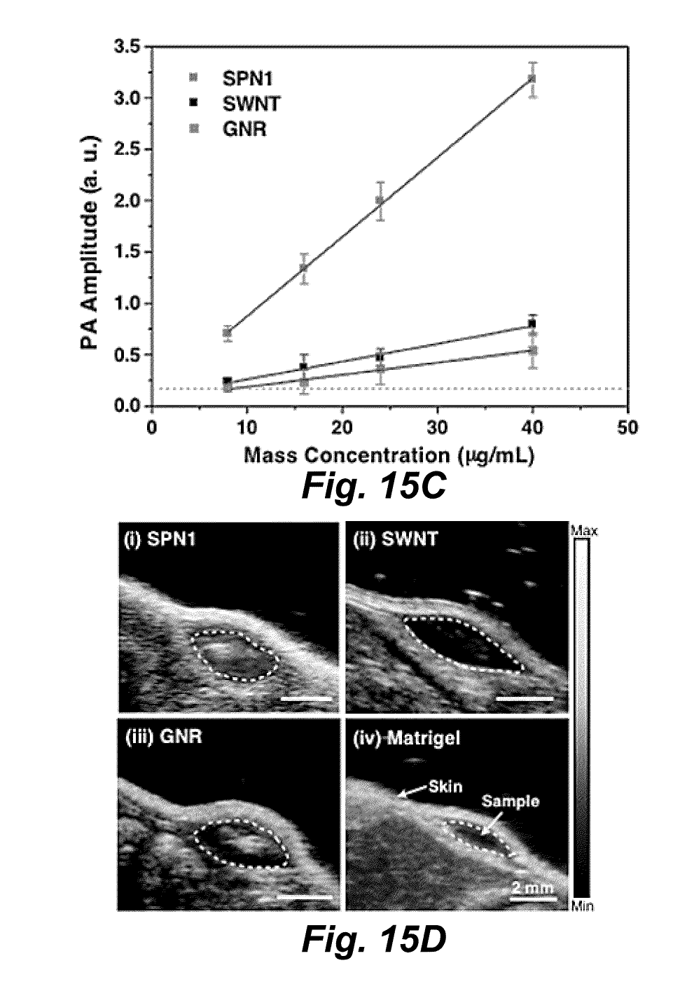

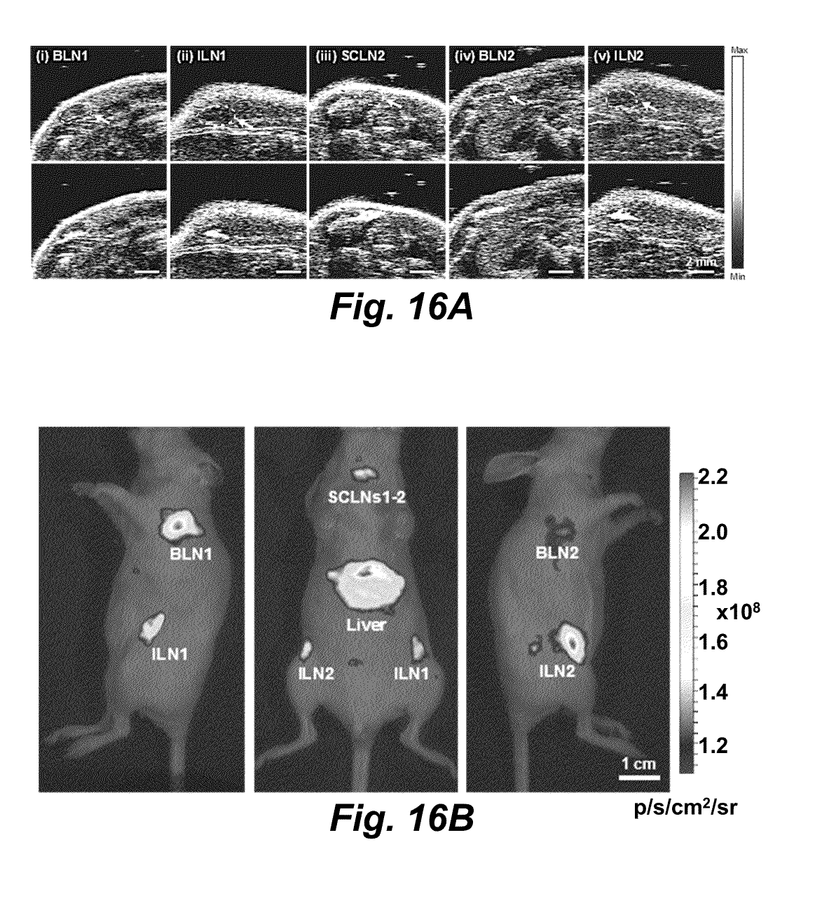

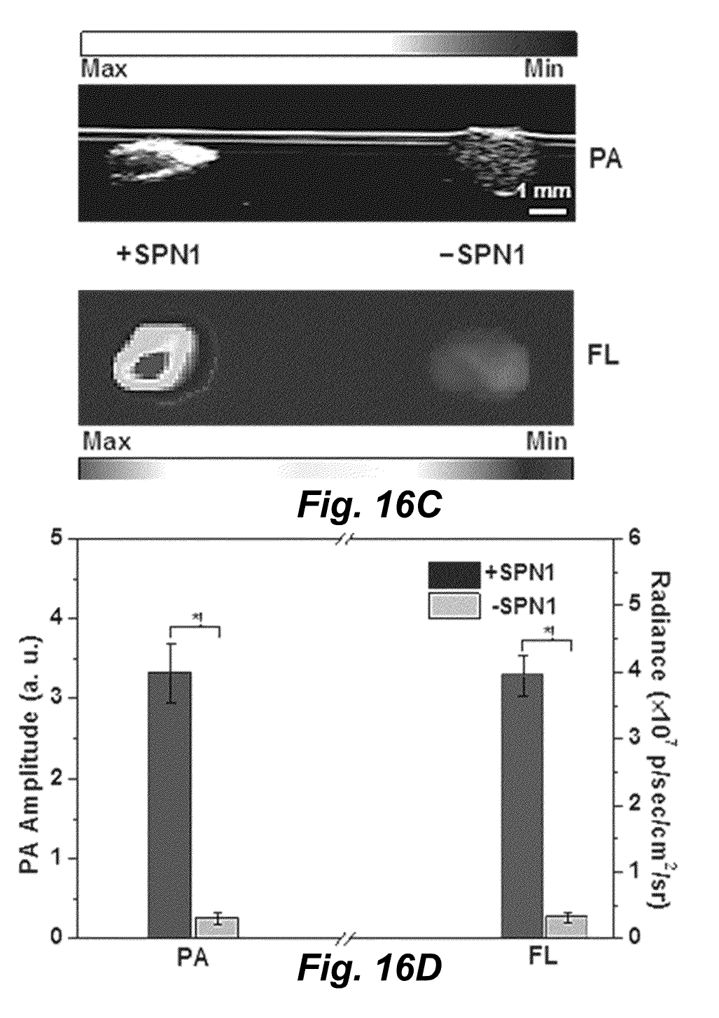

SPs such as those originally used to convert sunlight into electricity have now been made into nanoparticles that produce ultrasound signals upon pulsed near-infrared laser irradiation. These purely organic PA nanoparticles have a unique set of advantages that derive from their precursor SPs, including a large mass extinction coefficient and high photostability. These advantages make SPNs such as, for example, SPN1, SPN2, SPN3, SPN4, and SPN5, the structures of which are illustrated in FIGS. 1-5, respectively, superior PA imaging agents. They have stronger and more photostable PA signals in the NIR region when compared with single wall nanotubes (SWNT) and gold nanorods (GNR), as shown in FIGS. 15A-15D. At the same mass concentration, the intrinsic PA amplitude of PCPDTBT-based SPN (SPN1, shown in FIG. 1) at 700 nm was more than 5-times higher than SWNT and GNR (FIG. 15A). This increased PA signal generation resulted in an even lower limit of detection of SPN1 in living mice (2 μg/mL) relative to both SWNT and GNR (9 μg/mL) (FIG. 15D), ultimately leading to reduced dosing levels for in vivo PA imaging applications. This advantage in conjunction with its favorable size (approximately 40 nm) enabled the efficient PA imaging of major lymph nodes in living mice with a high signal-to-noise ratio of 13.3 after a single intravenous administration of a small amount of SPN1 (2.5 μg) as shown in FIGS. 16A-16D.

Although detailed toxicity investigations are required for clinical translation, the probes of the disclosure have showed no overt SPN cytotoxicity. Others have also reported that the implantation of SPs in living animals invoked little adverse tissue response with no evidence of acute and subacute toxicity. This level of biocompatibility was comparable to the FDA-approved polymer poly(lactic acid-co-glycolic acid) (Guimard et al., (2007) Prog. Polym. Sci. 32: 876-921), suggesting that they are advantageous for clinical applications.

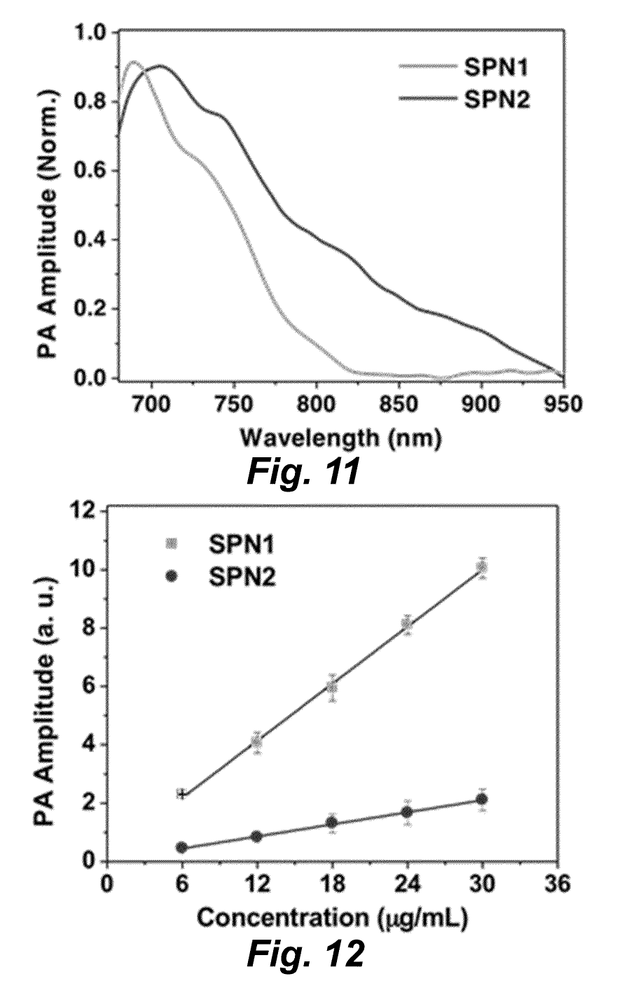

SPNs according to the disclosure are readily prepared through a nanoprecipitation-based bottom-up approach, and their PA properties can be easily adjusted by choosing different polymers from the library of photovoltaic SPs without significantly affecting particle dimensions. For example, SPN1 and SPN2 displayed similar sizes (approximately 40 nm) but different PA spectral profiles (see, for example, FIGS. 8 and 11). The independence of the photophysical properties from the size of SPNs is different from many of the properties of metallic nanoparticles where particle size directly modifies their spectral properties. This attribute of SPNs is beneficial to in vivo multiplexed imaging, as nanoparticles can be formulated to have distinct PA wavelengths with similar pharmacokinetic profiles. Additionally, other molecules such as dyes and drugs may be simultaneously doped into SPNs during nanoprecipitation, potentially endowing properties to SPN beyond simple PA imaging.

It is further contemplated that the SNPs according to the present disclosure may further include a therapeutic agent or combination of said agents that can modulate a physiological or biochemical process of a cell or tissue that is being imaged. Such agents include, but are not limited to, agents that can modulate the proliferation of a cancer or tumor cell. In some embodiments, the therapeutic agent can be an anti-inflammatory agent. In some embodiments the therapeutic agents that are combined with the SNPs can be a combination of more than one type of anti-cancer agent, more than one type of anti-inflammatory agent, or any combination thereof.

With synthetic and structural flexibility, SPN1 was developed into an activatable NIR ratiometric PA probe (RSPN) for ROS imaging in living animals. This study is the first demonstration of a PA imaging probe to detect ROS, since existing ROS probes are mainly based on fluorescence imaging and only a few have been tested in vivo (Dickinson et al., (2011) Nat. Chem. Biol. 7: 504-511; Nagano et al., (2009) J. Clin. Biochem. Nutr. 45: 111-124).

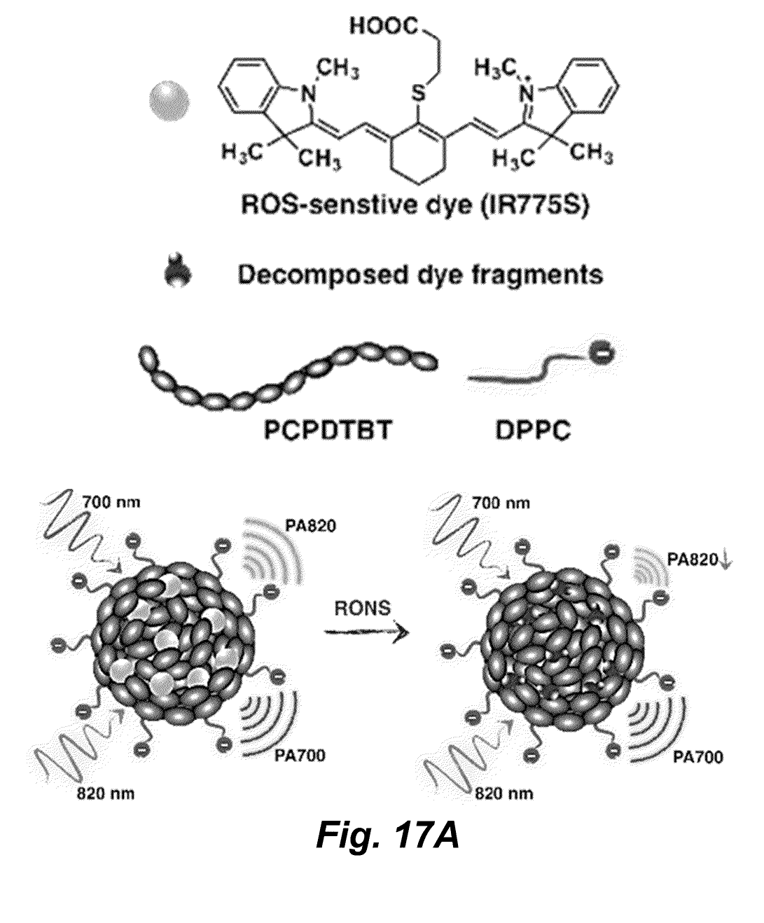

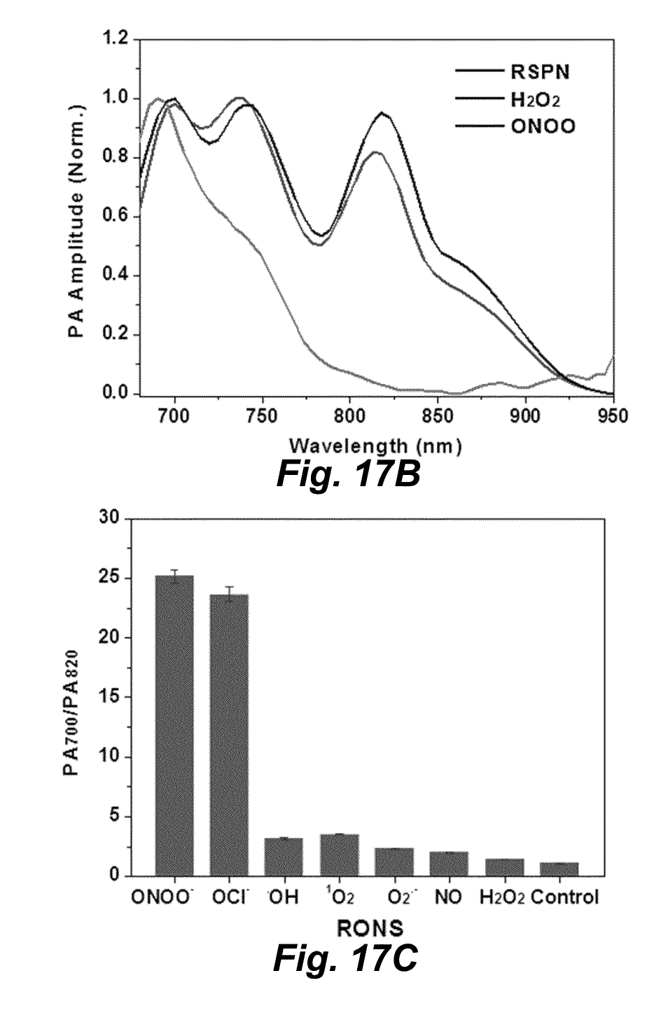

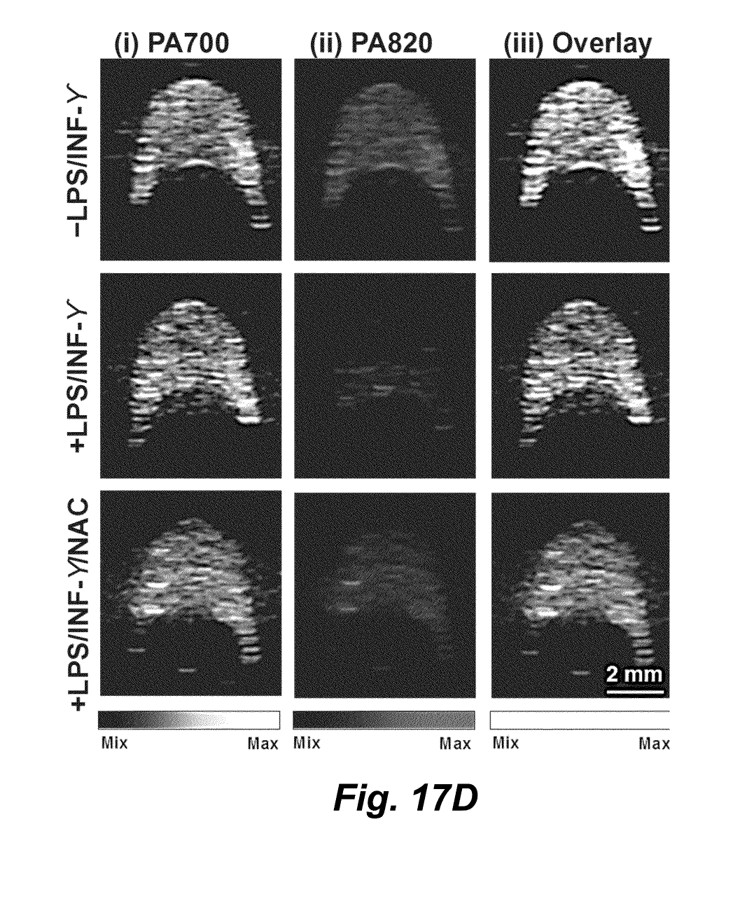

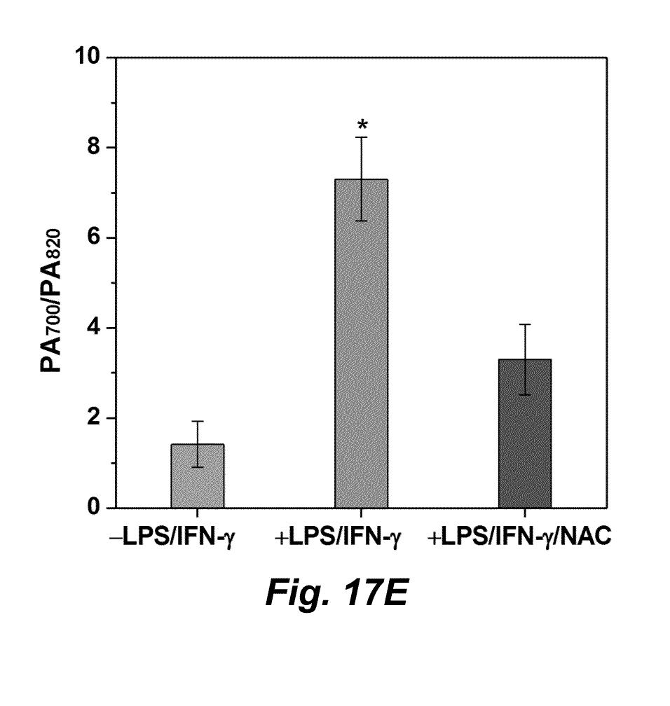

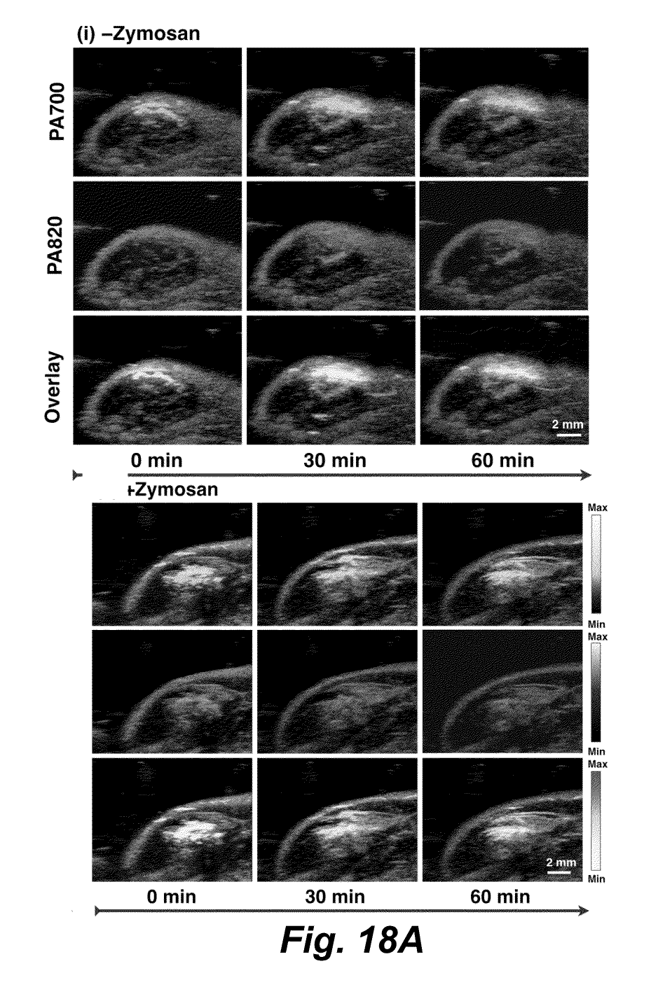

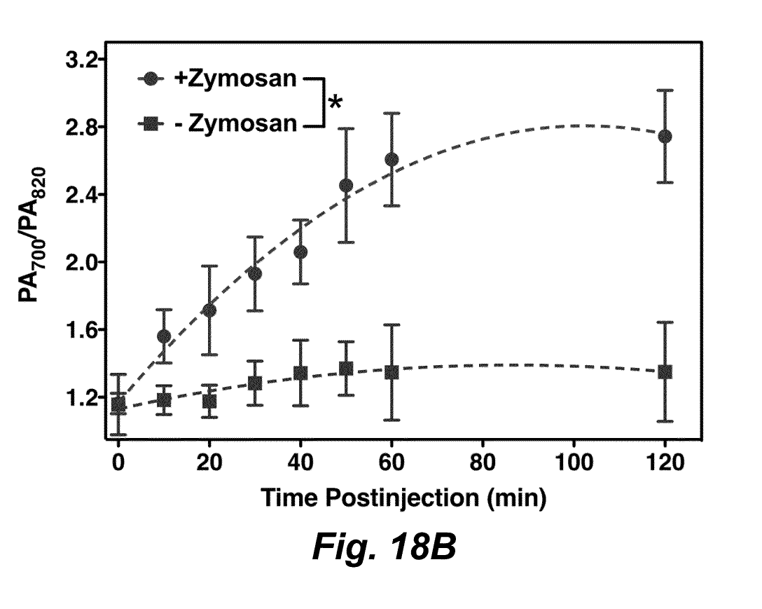

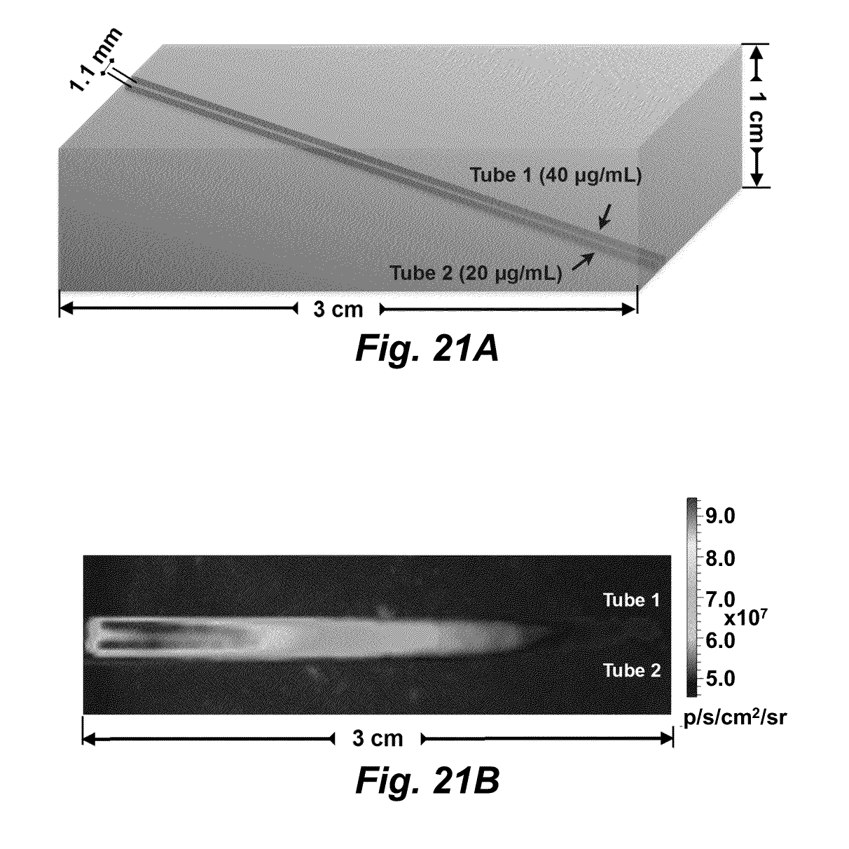

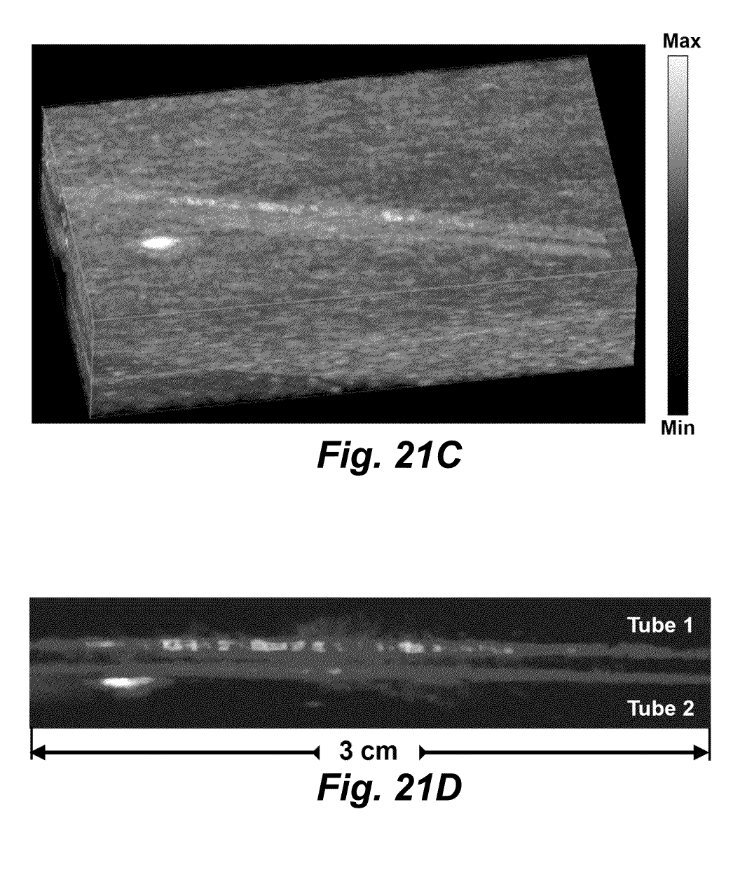

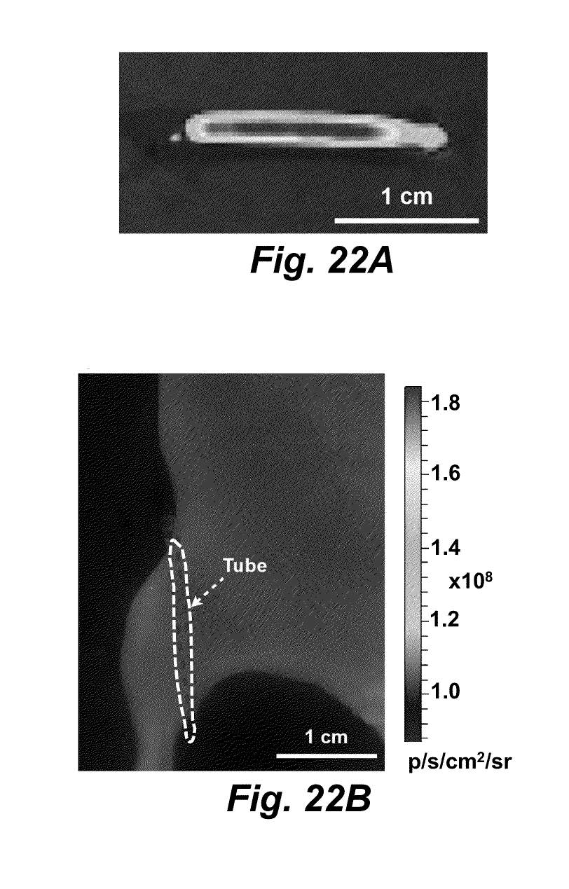

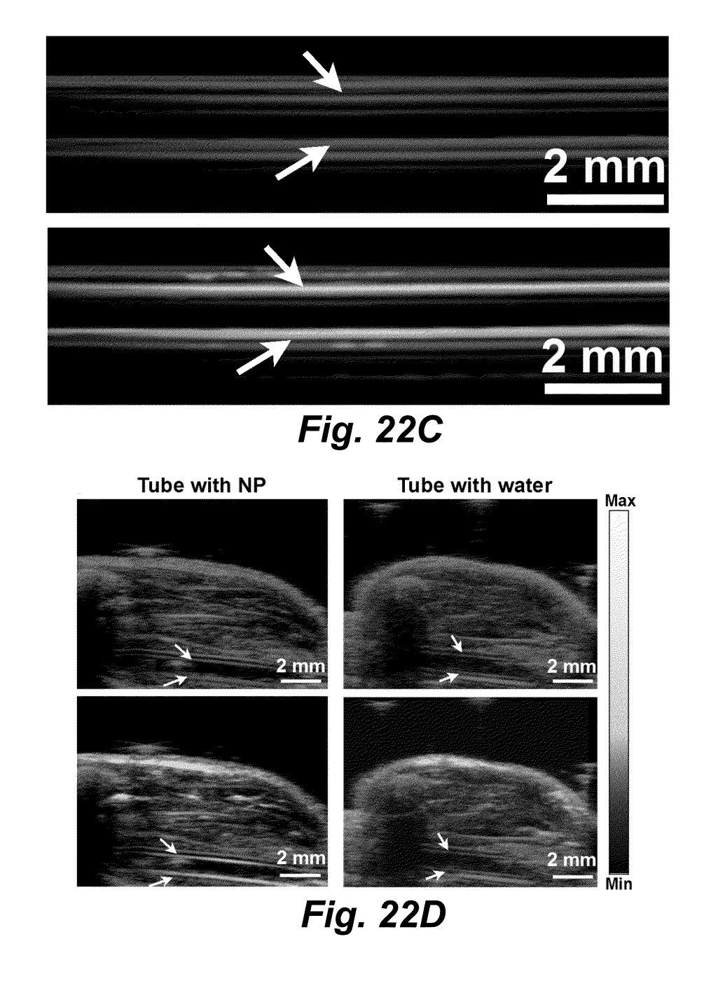

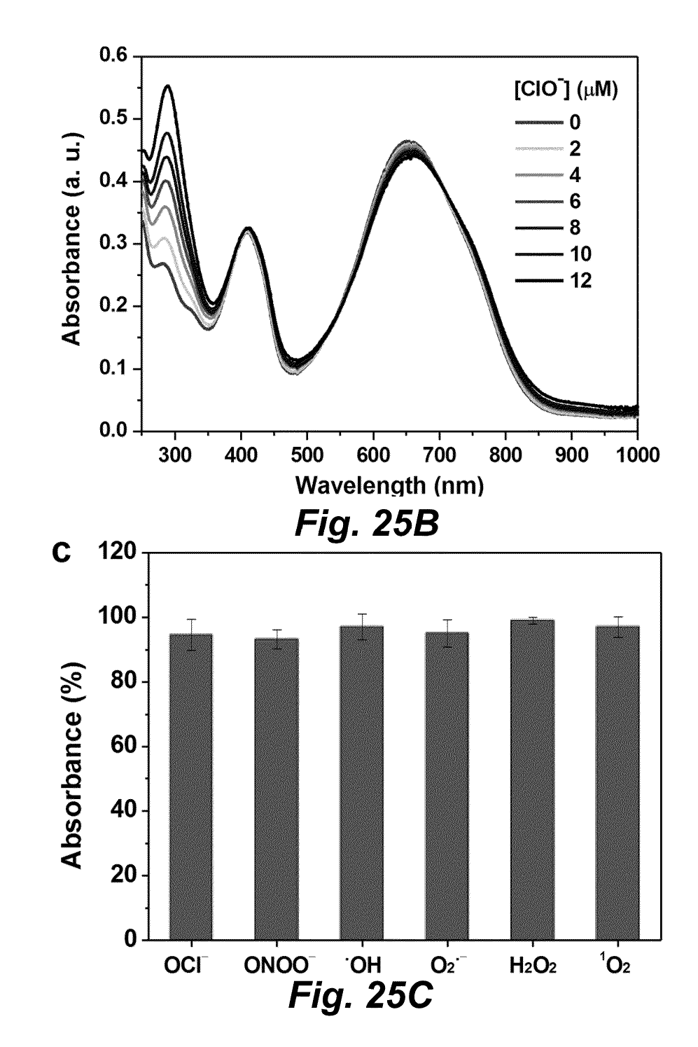

Characteristics of the SPNs according to the present disclosure such as a narrow PA spectral profile, good photostability, and an ROS-inert PA signal, make them unique among other nanoparticulate agents. Neither SWNT (planar PA spectrum) (De la Zerda et al., (2008) Nature Nanotech. 3: 557-562) nor GNR (poor photostability and poor stability to ROS, FIGS. 15B and 25A) possess all of these characteristics necessary for designing useful ratiometric ROS probes. Coupling of the excellent resistance to oxidation of SPN1 with the ROS sensing ability of IR775S, RSPN effectively detected ROS, and exhibited enhancements in ratiometric PA signals of 25, 7.3, and 2.7-times in solution (FIG. 17C), in cells (FIG. 17E) and in living mice (FIG. 18B), respectively. This level of sensitivity is comparable to, or even higher than, that reported for FL probes (Dickinson et al., (2011) Nat. Chem. Biol. 7: 504-511; Nagano et al., (2009) J. Clin. Biochem. Nutr. 45: 111-124). As PA imaging has the advantages of deep tissue penetration, high spatial resolution (FIGS. 21A-21D and 22A-22D), and the capability to simultaneously acquire anatomical and molecular information, RSPN have advantages over currently available FL probes, allowing interrogation of the key role of ROS in the etiology of diseases at levels not achievable by FL imaging.

Accordingly, the SPNs of the disclosure represent a new class of NIR PA contrast agent for in vivo PA molecular imaging. They can serve not only as simple accumulation-based PA probes (as demonstrated for lymph node mapping) but also as a platform to develop activatable probes (as shown by ratiometric PA imaging of inflammatory ROS in living mice). Given many key merits of SPNs such as strong signal amplitude, long-term photostability, size-independent spectral tunability, scalable and facile synthesis, compositional biocompatibility and structural diversity, the SPNs of the disclosure are advantageous for advanced PA molecular imaging for facilitating the preclinical investigation of physiological and pathological processes in living subjects.

One aspect of the disclosure encompasses embodiments of a nanoparticle comprising an organic photovoltaic semiconductor polymer and a phospholipid, where the semiconductor polymer is characterized as near-infra red absorbing and generating a detectable photoacoustic signal and a fluorescent emission when irradiated by an incident activation energy.

In some embodiments of this aspect of the disclosure, the semiconductor polymer can be poly(cyclopentadithiophene-alt-benzothiadiazole) (PCPDTBT), poly(acenaphthothienopyrazine-alt-benzodithiophene) (PATPBDT), poly[4,6-(dodecyl-thieno[3,4-b]thiophene-2-carboxylate)-alt-2,6-(4,8-dioctoxylbenzo[1,2-b:4,5-b]dit, poly[N-90-heptadecanyl-2,7carbazole-alt-3,6-bis(thiophen-5-yl)-2,5-dioctyl-2,5-dihydropyrrolo[3,4]pyrrole-1,4-dione], poly{4,8-bis[(2-ethylhexyl)oxy]benzo[1,2-b:4,5-b′]dithiophene-2,6-diyl-alt-3-fluoro-2-[(2-ethylhexyl], poly[2,6(4,4′bis(ethylhexyl)dithieno[3,2-b:2′,3′-d]siloleyalt-(1,3-(5-octyl-4H-thieno[3,4-c]pyrrole], and the like.

In some embodiments of this aspect of the disclosure, the semiconductor polymer can be selected from the group consisting of the polymers designated as SNP1, SNP2, SNP3, SNP4, and SNP5.

In some embodiments of this aspect of the disclosure, the phospholipid can be 1,2-dipalmitoyl-sn-glycero-3-phosphocholine (DPPC), 1,2-dinonadecanoyl-sn-glycero-3-phosphocholine, 1,2-diarachidoyl-sn-glycero-3-phosphocholine, 1,2-dibehenoyl-sn-glycero-3-phosphocholine, 1,2-dilignoceroyl-sn-glycero-3-phosphocholine, 1,2-distearoyl-sn-glycero-3-phosphoethanolamine-N-[methoxy(polyethylene glycol)-2000] (ammonium salt), 1,2-dioleoyl-sn-glycero-3-phosphoethanolamine-N-[methoxy(polyethylene glycol)-2000] (ammonium salt), 1,2-dimyristoyl-sn-glycero-3-phosphoethanolamine-N-[methoxy(polyethylene glycol)-350] (ammonium salt), 1,2-distearoyl-sn-glycero-3-phosphoethanolamine-N-[methoxy(polyethylene glycol)-550] (ammonium salt), 1,2-distearoyl-sn-glycero-3-phosphoethanolamine-N-[methoxy(polyethylene glycol)-3000] (ammonium salt), or 1,2-distearoyl-sn-glycero-3-phosphoethanolamine-N-[methoxy(polyethylene glycol)-5000] (ammonium salt).

In embodiments of this aspect of the disclosure, the nanoparticle can further comprise a reactive oxygen species (ROS)-inactivated fluorophore.

In some embodiments of this aspect of the disclosure, the fluorophore can be 2-[4′-(β-carboxyethylthio)-7′-(1″,3″,3″-trimethylindolenine)-3′,5′-trimethyleneheptatrien-1-yl]-1,3,3-trimethylindolenium perchlorate (IR775S).

In embodiments of this aspect of the disclosure, the nanoparticle can further comprise at least one therapeutic agent.

Another aspect of the disclosure encompasses embodiments of a pharmaceutically acceptable composition comprising a nanoparticle comprising an organic photovoltaic semiconductor polymer and a phospholipid, wherein the semiconductor polymer is characterized as near-infra red absorbing and generating a detectable photoacoustic signal and a fluorescent emission when irradiated by an incident activation energy.

In some embodiments of this aspect of the disclosure, the semiconductor polymer can be poly(cyclopentadithiophene-alt-benzothiadiazole) (PCPDTBT), poly(acenaphthothienopyrazine-alt-benzodithiophene) (PATPBDT), poly[4,6-(dodecyl-thieno[3,4-b]thiophene-2-carboxylate)-alt-2,6-(4,8-dioctoxylbenzo[1,2-b:4,5-b]dit, poly[N-90-heptadecanyl-2,7carbazole-alt-3,6-bis(thiophen-5-yl)-2,5-dioctyl-2,5-dihydropyrrolo[3,4]pyrrole-1,4-dione], poly{4,8-bis[(2-ethylhexyl)oxy]benzo[1,2-b:4,5-b′]dithiophene-2,6-diyl-alt-3-fluoro-2-[(2-ethylhexyl], poly[2,6(4,4′bis(ethylhexyl)dithieno[3,2-b:2′,3′-d]siloleyalt-(1,3-(5-octyl-4H-thieno[3,4-c]pyrrole].

In some embodiments of this aspect of the disclosure, the semiconductor polymer can be selected from the group consisting of the polymers designated as SNP1, SNP2, SNP3, SNP4, and SNP5.

In some embodiments of this aspect of the disclosure, the phospholipid can be 1,2-dipalmitoyl-sn-glycero-3-phosphocholine (DPPC), 1,2-dinonadecanoyl-sn-glycero-3-phosphocholine, 1,2-diarachidoyl-sn-glycero-3-phosphocholine, 1,2-dibehenoyl-sn-glycero-3-phosphocholine, 1,2-dilignoceroyl-sn-glycero-3-phosphocholine, 1,2-distearoyl-sn-glycero-3-phosphoethanolamine-N-[methoxy(polyethylene glycol)-2000] (ammonium salt), 1,2-dioleoyl-sn-glycero-3-phosphoethanolamine-N-[methoxy(polyethylene glycol)-2000] (ammonium salt), 1,2-dimyristoyl-sn-glycero-3-phosphoethanolamine-N-[methoxy(polyethylene glycol)-350] (ammonium salt), 1,2-distearoyl-sn-glycero-3-phosphoethanolamine-N-[methoxy(polyethylene glycol)-550] (ammonium salt), 1,2-distearoyl-sn-glycero-3-phosphoethanolamine-N-[methoxy(polyethylene glycol)-3000] (ammonium salt), or 1,2-distearoyl-sn-glycero-3-phosphoethanolamine-N-[methoxy(polyethylene glycol)-5000] (ammonium salt).

In some embodiments of this aspect of the disclosure, the nanoparticle can further comprise a reactive oxygen species (ROS)-inactivated fluorophore.

In some embodiments of this aspect of the disclosure, the fluorophore can be 2-[4′-(β-carboxyethylthio)-7′-(1″,3″,3″-trimethylindolenine)-3′,5′-trimethyleneheptatrien-1-yl]-1,3,3-trimethylindolenium perchlorate (IR775S).

In embodiments of this aspect of the disclosure, the pharmaceutically acceptable composition can further comprise a pharmaceutically acceptable carrier.

In embodiments of this aspect of the disclosure, the nanoparticle can further comprise at least one therapeutic agent.

Yet another aspect of the disclosure encompasses embodiments of a method of molecular imaging, comprising the steps of: (a) delivering to a human or non-human subject a pharmaceutically acceptable composition comprising a plurality of nanoparticles comprising an organic photovoltaic semiconductor polymer, and a phospholipid a reactive oxygen species (ROS)-inactivated fluorophore, wherein the semiconductor polymer is characterized as near-infra red absorbing and generating a first detectable photoacoustic signal spectrum when irradiated by an incident activation energy; (b) irradiating the human or non-human subject with a first incident energy having a first wavelength and generating a first photoacoustic signal; (c) irradiating the human or non-human subject with a second incident energy having a second wavelength and generating a second photoacoustic signal; (d) determining the intensities of the first and the second photoacoustic signals; (e) determining a first ratio of the intensities of the first and the second photoacoustic signals; (f) comparing said first ratio with a second ratio determined from the nanoparticles before delivery to the human or non-human subject, whereby a difference in said first and second ratios indicates RONS degradation of the nanoparticles in the subject; and (g) generating a ratiometric image indicating the difference in said first and second ratios relative to an image of the subject.

In some embodiments of this aspect of the disclosure, the semiconductor polymer can be poly(cyclopentadithiophene-alt-benzothiadiazole) (PCPDTBT), poly(acenaphthothienopyrazine-alt-benzodithiophene) (PATPBDT), poly[4,6-(dodecyl-thieno[3,4-b]thiophene-2-carboxylate)-alt-2,6-(4,8-dioctoxylbenzo[1,2-b:4,5-b]dit, poly[N-90-heptadecanyl-2,7carbazole-alt-3,6-bis(thiophen-5-yl)-2,5-dioctyl-2,5-dihydropyrrolo[3,4]pyrrole-1,4-dione], poly{4,8-bis[(2-ethylhexyl)oxy]benzo[1,2-b:4,5-b′]dithiophene-2,6-diyl-alt-3-fluoro-2-[(2-ethylhexyl], poly[2,6(4,4′bis(ethylhexyl)dithieno[3,2-b:2′,3′-d]siloleyalt-(1,3-(5-octyl-4H-thieno[3,4-c]pyrrole].

In some embodiments of this aspect of the disclosure, the semiconductor polymer can be selected from the group consisting of the polymers designated as SNP1, SNP2, SNP3, SNP4, and SNP5.

In some embodiments of this aspect of the disclosure, the phospholipid can be 1,2-dipalmitoyl-sn-glycero-3-phosphocholine (DPPC), 1,2-dinonadecanoyl-sn-glycero-3-phosphocholine, 1,2-diarachidoyl-sn-glycero-3-phosphocholine, 1,2-dibehenoyl-sn-glycero-3-phosphocholine, 1,2-dilignoceroyl-sn-glycero-3-phosphocholine, 1,2-distearoyl-sn-glycero-3-phosphoethanolamine-N-[methoxy(polyethylene glycol)-2000] (ammonium salt), 1,2-dioleoyl-sn-glycero-3-phosphoethanolamine-N-[methoxy(polyethylene glycol)-2000] (ammonium salt), 1,2-dimyristoyl-sn-glycero-3-phosphoethanolamine-N-[methoxy(polyethylene glycol)-350] (ammonium salt), 1,2-distearoyl-sn-glycero-3-phosphoethanolamine-N-[methoxy(polyethylene glycol)-550] (ammonium salt), 1,2-distearoyl-sn-glycero-3-phosphoethanolamine-N-[methoxy(polyethylene glycol)-3000] (ammonium salt), or 1,2-distearoyl-sn-glycero-3-phosphoethanolamine-N-[methoxy(polyethylene glycol)-5000] (ammonium salt).

In some embodiments of this aspect of the disclosure, the fluorophore can be 2-[4′-(β-carboxyethylthio)-7′-(1″,3″,3″-trimethylindolenine)-3′,5′-trimethyleneheptatrien-1-yl]-1,3,3-trimethylindolenium perchlorate (IR775S).

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### EXAMPLE

### Example 1

### Synthesis

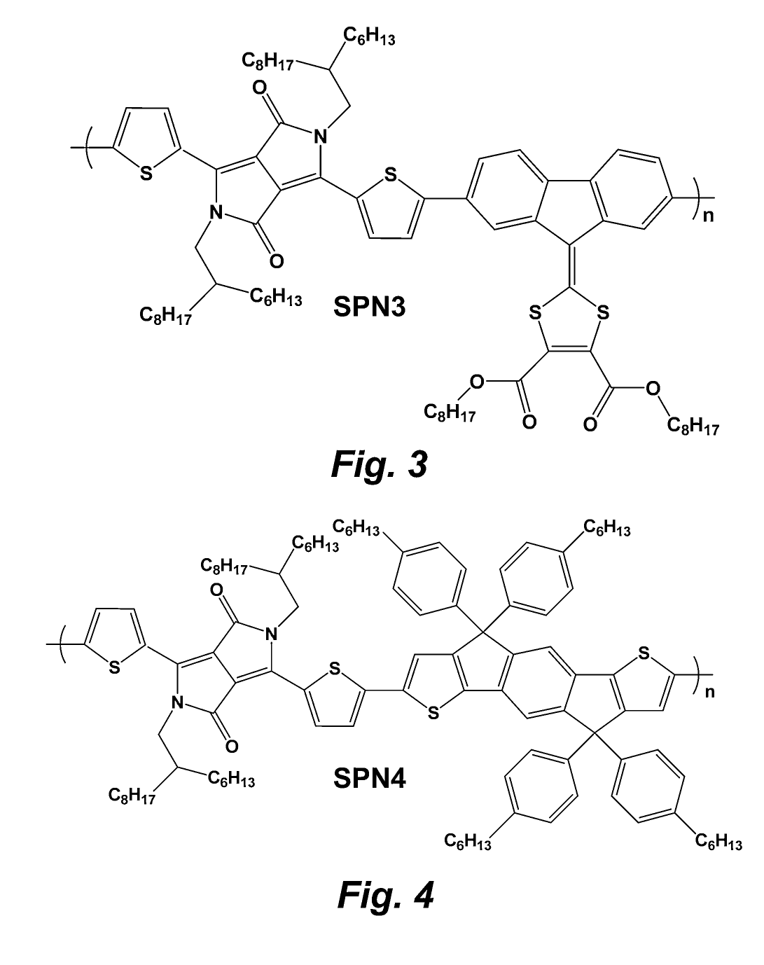

Two efficient photovoltaic SPs with strong absorption in the NIR region, poly(cyclopentadithiophene-alt-benzothiadiazole) (PCPDTBT, FIG. 1) (Muhlbacher et al., (2006) Adv. Mater. 18: 2884-2889) and poly(acenaphthothienopyrazine-alt-benzodithiophene) (PATPBDT) derivatives (FIG. 2) (Mei et al., (2013) J. Am. Chem. Soc. 135: 6724-6746), were used to prepare PA SPNs. In addition diketopyrrolopyrroles were also used to generate the SNPs designated as SPN3, SPN4, and SPN5, the structures of which are illustrated in FIGS. 3-5, respectively).

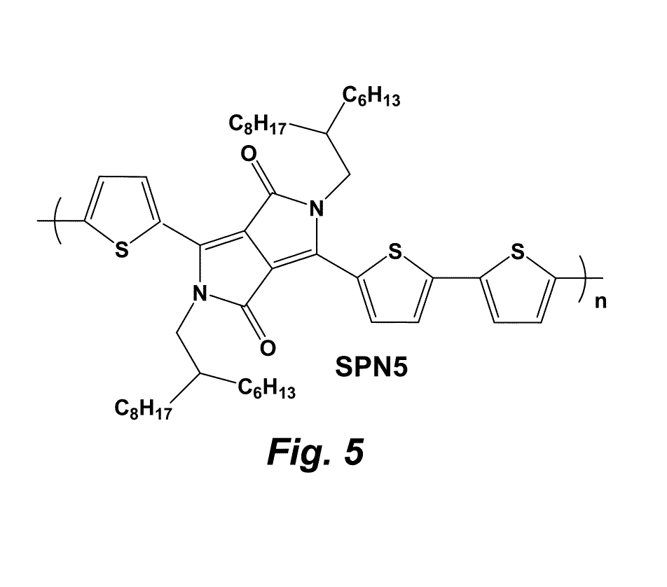

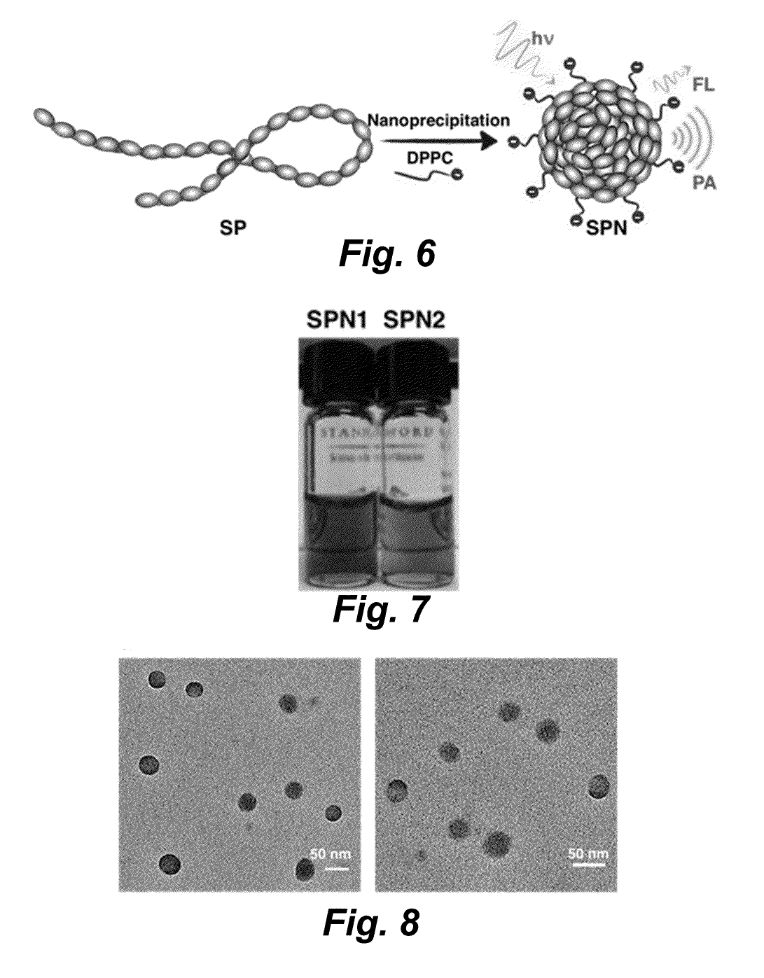

For example, nanoprecipitation assisted by 1,2-dipalmitoyl-sn-glycero-3-phosphocholine (DPPC) (FIG. 5) afforded water-soluble PCPDTBT-based (SPN1) and PATPBDT-based (SPN2) nanoparticles as clear dark-green and olive-green solutions (FIG. 6), respectively.

### Example 2

### Characterization

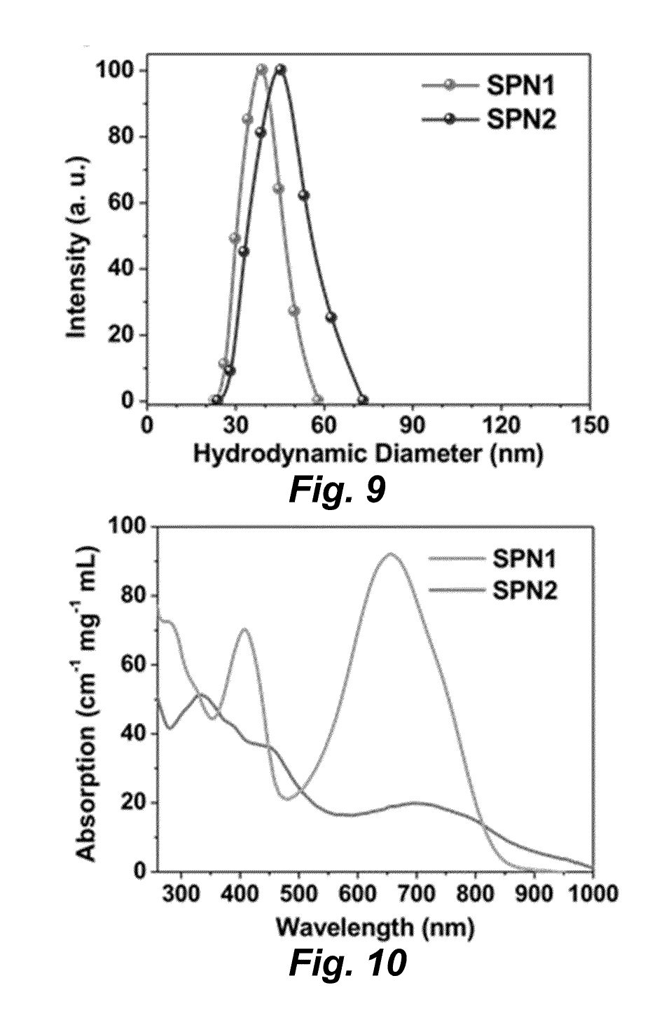

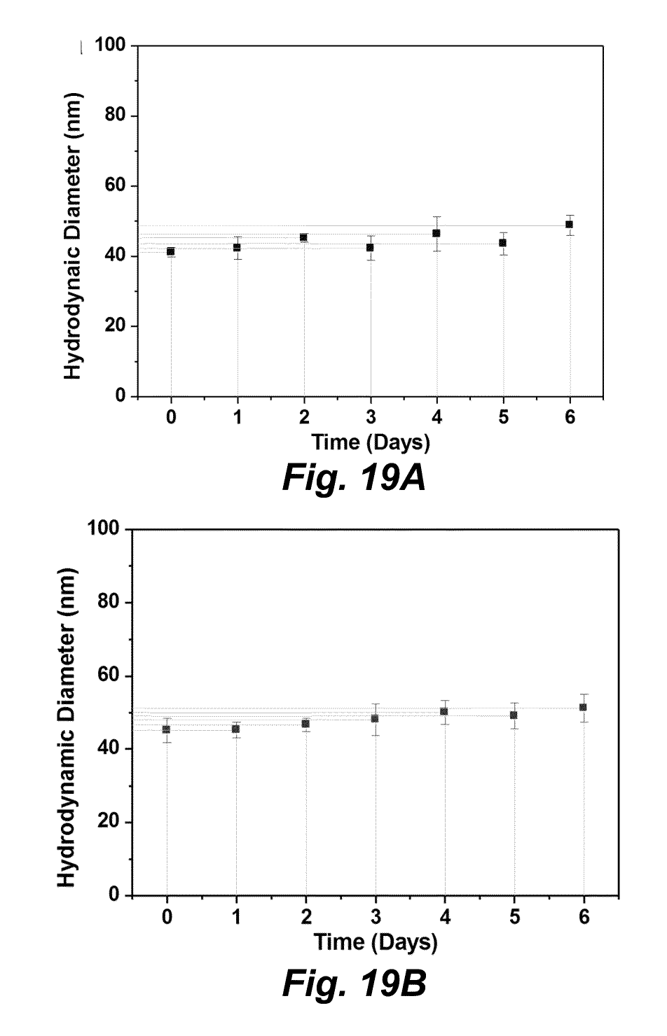

Transmission electron microscopy (TEM) revealed that SPN1 and SPN2 had spherical morphologies with average diameters of 41±2 and 43±2 nm, respectively (as shown in FIG. 8). Moreover, dynamic light scattering (DLS) confirmed their narrow size distribution with polydispersity indexes of 0.16±0.03 and 0.19±0.02 for SPN1 and SPN2, respectively (FIG. 9). The zeta potentials of SPN1 and SPN2 were −35±2 and −32±3 mV in PBS (pH=7.4), respectively. Such large zeta potentials were attributed to the presence of DPPC, which resulted in high SPN stability under physiologically relevant conditions with no obvious size change for both SPNs at least one week after synthesis (FIGS. 19A and 19B).

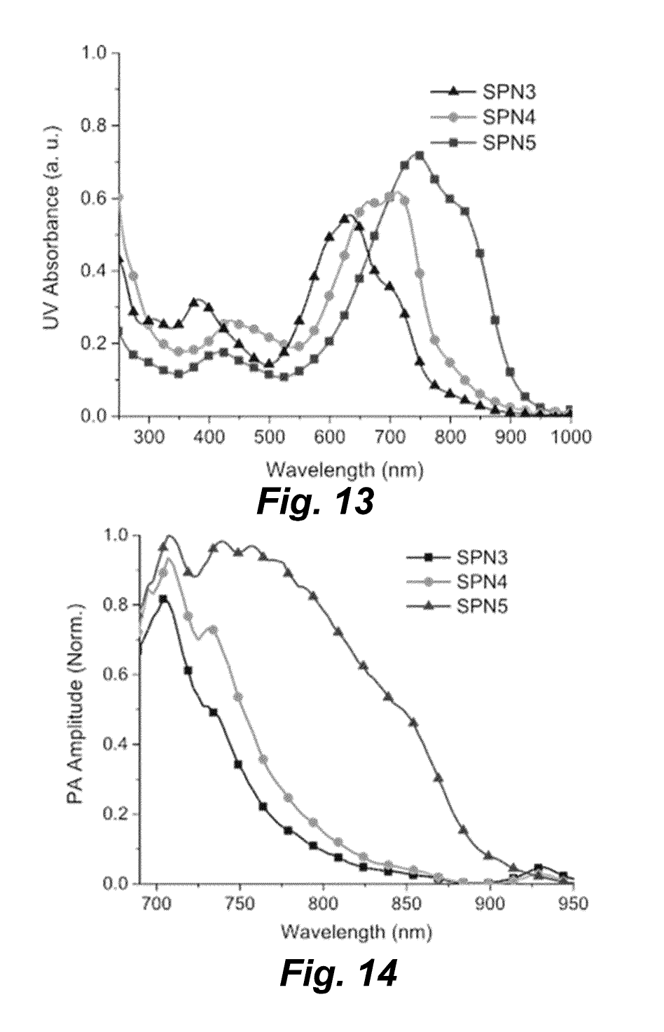

SPN1 and SPN2 had NIR absorptions with maxima at 660 and 700 nm (FIG. 10), respectively. The SPNs designated SPN3, SPN4, and SPN5 had absorption peaks of about 625 nm, about 650 nm to about 725 nm, and about 750 nm, respectively as shown in FIG. 13.

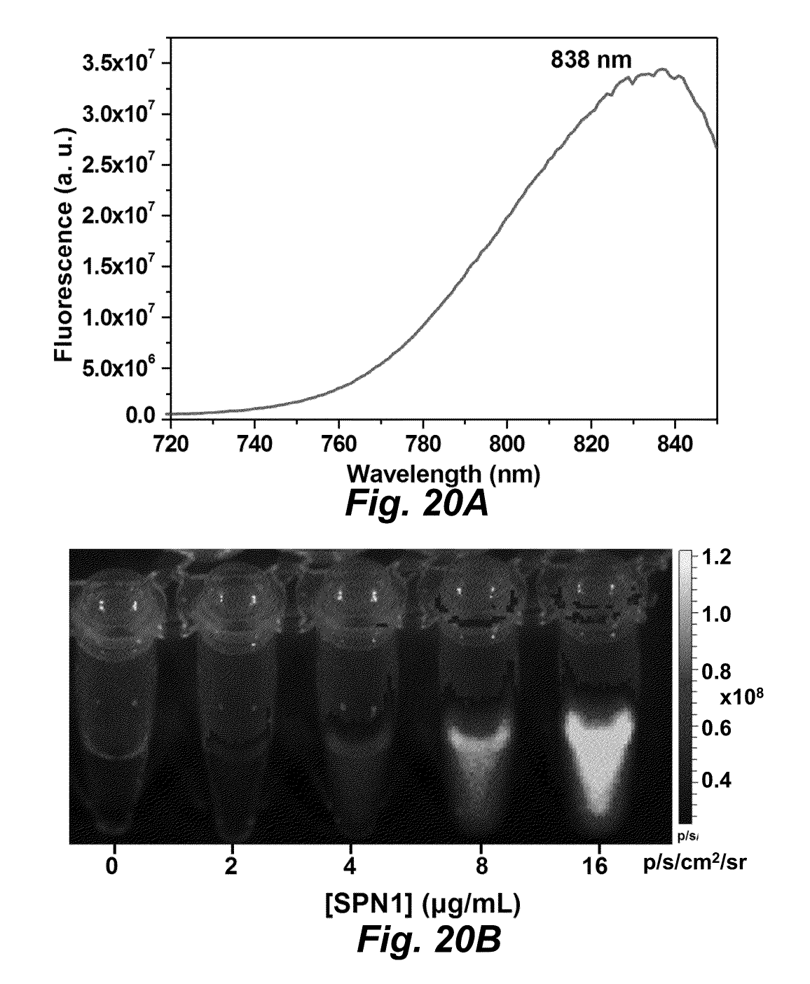

Due to the difference in the molecular structures of PCPDTBT and PATPBDT, the peak mass extinction coefficient of SPN1 (93 cm⁻¹ mγ-¹ mL) was 4.65-times higher than that of SPN2 (20 cm⁻¹ my-¹ mL). While SPN1 was fluorescent upon excitation at 680 nm with emission maximum at 838 nm and a quantum yield of 0.1% in 1×PBS at pH=7.4 (FIGS. 20A and 20B), SPN2 did not show detectable FL below 900 nm.

With a strong absorption in the NIR region, both SPNs efficiently generated PA signals upon pulsed laser irradiation ranging from 680 to 825 nm for SPN1, SPN3, and SPN4, and 680 to 950 nm for SPN2 and SPN5 (FIGS. 11 and 14). The narrower PA spectrum profile for SPN1 was caused by its narrower absorption above 600 nm relative to SPN2 (FIG. 10). The maximum PA signals were observed at 690 and 705 nm for SPN1 and SPN2, respectively, both of which were red-shifted as compared to their maximum absorption. It is well known that the absorption of SPs results from heterogeneous polymer segments with different Tr-conjugation lengths and π-π stacking strengths (Schwartz et al., (2003) Annu. Rev. Phys. Chem. 54: 141-172). Strong π-π stacking favors nonradiative deactivation that releases photoexcitation energy in the form of heat. Since the PA amplitude is mainly determined by heat generation, the segments within SPNs with stronger π-π stacking and longer absorption wavelength contribute more to heat generation. This increased sensitivity of PA to π-π stacking underlies the origin of the red-shifted PA maxima vs. the UV-Vis absorption maxima.

The PA amplitudes of both SPNs at 700 nm were determined at different concentrations in an agar phantom. A linear relationship between PA signal and concentration was found for SPN1 and SPN2, with respective R² of 0.998 and 0.997 (FIG. 12). Because of the larger extinction coefficient, the PA amplitude of SPN1 was 5-times higher than that of SPN2 at all concentrations. The narrower PA spectral profile and higher PA amplitude of SPN1 as compared with those of SPN2 make it a better platform for the construction of ratiometric PA molecular imaging probes.

The capability of SPN1 to produce both FL and PA signals enabled a direct intermodality comparison of PA with FL imaging in terms of SPN performance. In a tissue-mimicking gelatin phantom containing hemoglobin and intralipid, two polyethylene (PE) tubes filled with SPN1 at different concentrations were clearly visualized by PA imaging with nearly no signal attenuation up to a depth of 1 cm, as shown in FIGS. 16A-16D. However, neither tube was distinguished by FL imaging, with significant signal loss within 0.5 cm of imaging depth. More importantly, PA imaging clearly delineated the SPN1-filled PE tube through overlying muscle, skin, and connective tissue of the intact thigh of a living mouse, but no signal was detectable for FL imaging, as shown in FIGS. 17A-17D. Moreover, the inner diameter of the tube was measured to be 0.70±0.02 mm from the PA images both in vitro and in vivo, which matched with its actual value (0.70 mm). Thus, PA imaging with SPN1 can provide better spatial resolution and much larger penetration depths relative to FL imaging.

### Example 3

Comparison with SWNT and GNR: The PA properties of SPNs were further evaluated by comparing SPN1 with currently the best exogenous PA contrast agents, SWNT and GNR (De la Zerda et al., (2008) Nature Nanotech. 3: 557-562; Jokerst et al., (2012) ACS Nano 6: 10366-10377), in both agar phantom and living mice. The SWNT produced a strong PA amplitude at 690 nm and the GNR had a PA maximum at 705 nm (De la Zerda et al., (2008) Nature Nanotech. 3: 557-562; Jokerst et al., (2012) ACS Nano 6: 10366-10377), which were similar to SPN1 (690 nm) and thus permitted comparison with a pulsed laser at 700 nm (as shown in FIGS. 15A-15D).

When compared at the same mass concentration, SPN1 had the highest PA amplitude among these nanoparticles (FIG. 15A), which was 5.2 and 7.1-times higher than SWNT and GNR, respectively. Although this result was qualitatively consistent with the rank order of their mass extinction coefficients (93, 50 and 45 cm⁻¹ mg-¹ mL for SPN1, SWNT and GNR, respectively), the large differences in their PA signal amplitude suggested that additional mechanisms, such as the difference in heat conductance and heat capacity, should be relevant.

If calculated at equal molar concentrations, GNR provided the highest PA amplitude, as it had the largest molar mass among these nanoparticles (FIG. 15A). However, after continuous pulsed laser irradiation for 20 min, the PA signal of GNR decreased by 30±3% (FIG. 15B), which indicated the poor stability of GNR caused by laser-induced deformation (Link et al., (2000) J. Phys. Chem. B 104: 6152-6163). In contrast, no PA signal loss was observed for SPN1 under the same conditions, revealing its superior photostability and suitability for long-term PA molecular imaging.

The ability to detect PA signal from the nanoparticles in living subjects was tested and compared through subcutaneous injections of matrigel-containing solutions of SPN1, SWNT or GNR at different concentrations into the dorsal area of living mice. Linear correlations between the concentration and the PA signal were observed for all nanoparticles (FIG. 15C). At each concentration, the PA amplitude of SPN1 was approximately 4.0 and 5.8-times higher than SWNT and GNR, respectively, which were similar to that in agar phantoms. The limit of detection of SPN1 in living mice was calculated to be approximately 2 μg/mL (3.8 nM). The PA/ultrasound (US) co-registered images show that neither SWNT nor GNR was detectable at a concentration of 8 μg/mL (FIG. 15D), with PA signals approaching that of tissue background as calculated from the average PA signal in areas free from nanoparticles. This coincides with previous reports that set the limit of detection for SWNT and GNR in living mice by PA tomography close to 9 μg/mL (Eghtedari et al., (2007) Nano Letters 7: 1914-1918; Kim et al., (2009) Nature Nanotech. 4: 688-694; De la Zerda et al., (2008) Nature Nanotech. 3: 557-562).

### Example 4

### PA Imaging of Lymph Nodes

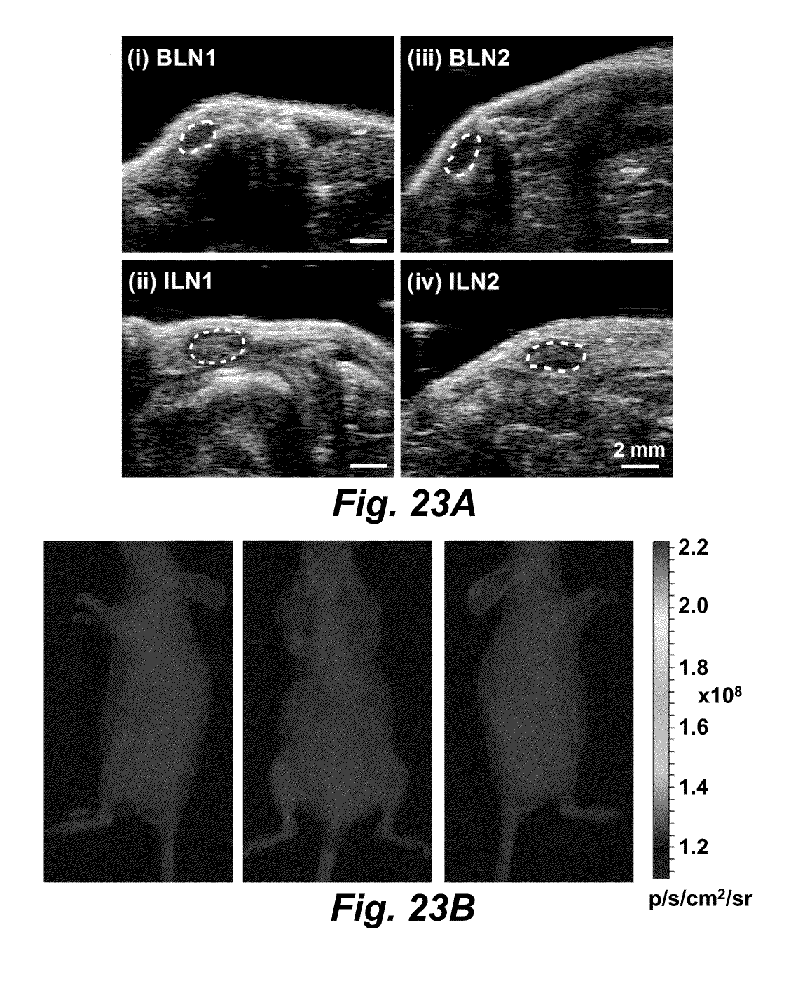

Nanoparticles with a diameter in the range of 20 to 50 nm have favorable accumulation and retention in draining lymph nodes, and thus are promising for lymph node tracking, which is clinically important to guide surgical resection of tumor tissues (Kim et al., (2004) Nat. Biotechnol. 22: 93-97; Nakajima et al., (2005) Cancer Sci. 96: 353-356). SPN1 (50 μg) were administered to healthy mice (n=4) intravenously (i.v.) for lymph node imaging. At 24 h post-injection, the mice were imaged with both PA and FL modalities (FIGS. 16A and 16B). Strong PA and FL signals were detected in the lymphatic networks of living mice, including accumulation in brachial lymph nodes (BLNs), inguinal lymph nodes (ILNs) and superficial cervical lymph nodes (SCLNs), which were undetectable prior to nanoparticle administration, as shown in FIGS. 23A and 23B.

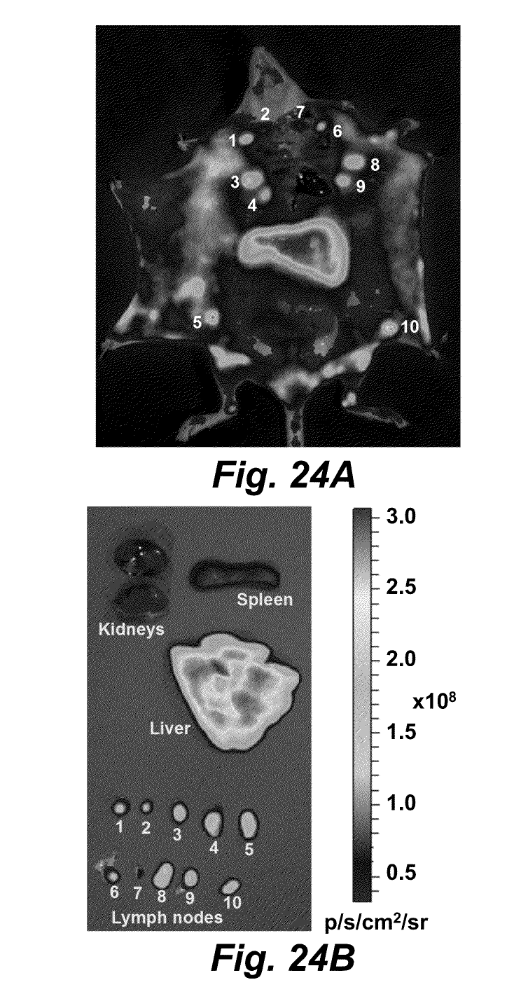

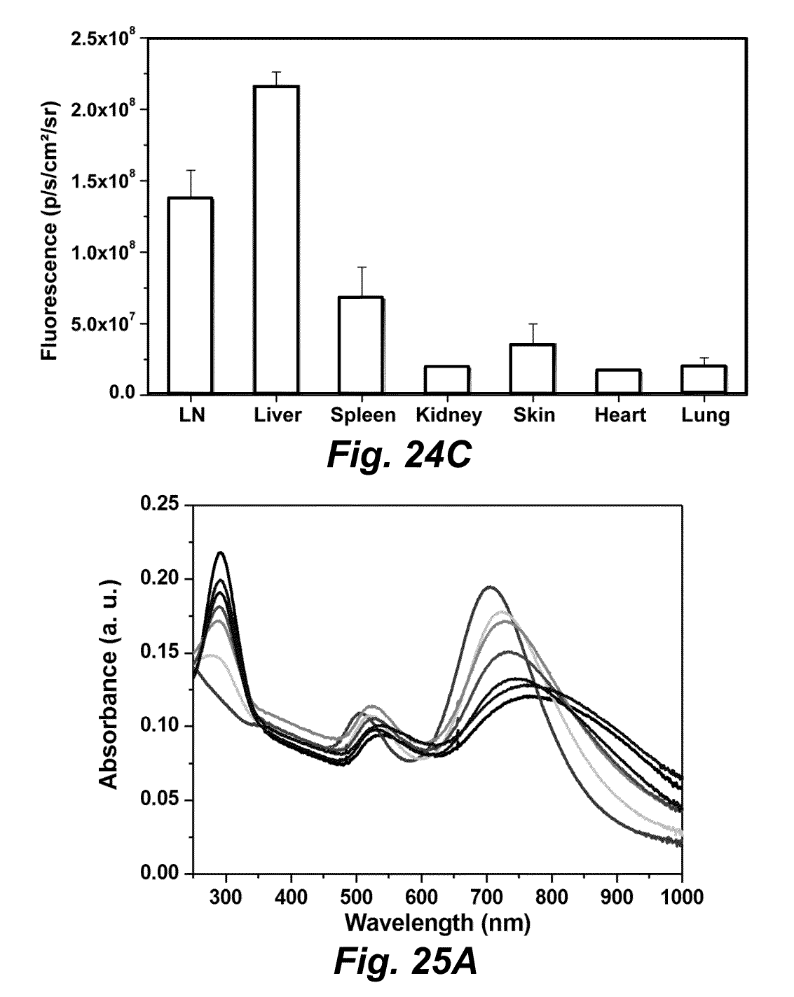

The high accumulation of SPN1 in the lymph nodes was confirmed following necropsy, showing that the second highest nanoparticle accumulation occurred in the lymph nodes, with an intensity equal to 63% of that from liver, as shown in FIGS. 24A-24C. Ex vivo lymph node imaging in an agar phantom showed a 13.3-times enhancement in PA signal following SPN administration relative to LN from a mouse receiving saline (FIGS. 16C and 16D), which was similar to the enhancement in observed FL signal (12.3-times). This result demonstrates that SPN1 can be used for lymph node mapping with both PA and FL imaging.

### Example 5

Ratiometric PA Imaging of ROS-Synthesis of RSPNs:

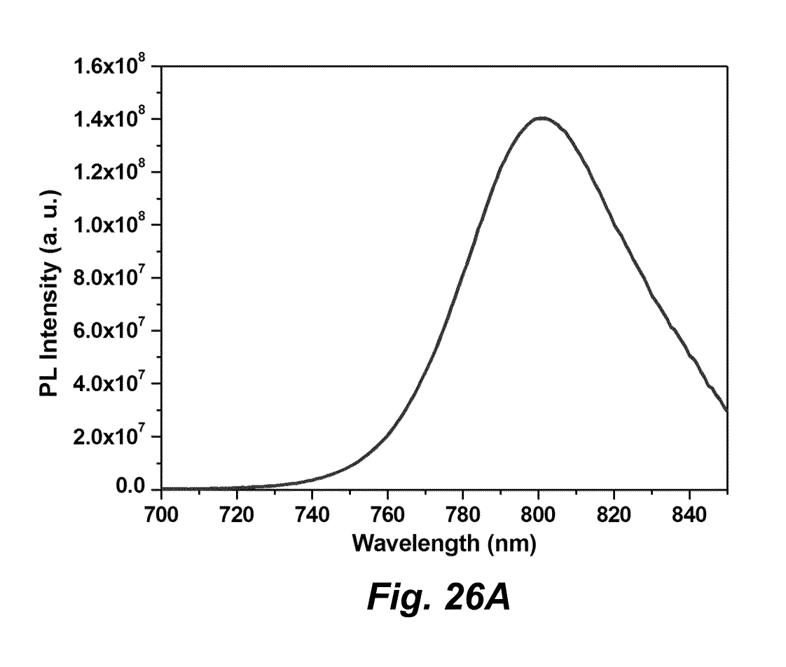

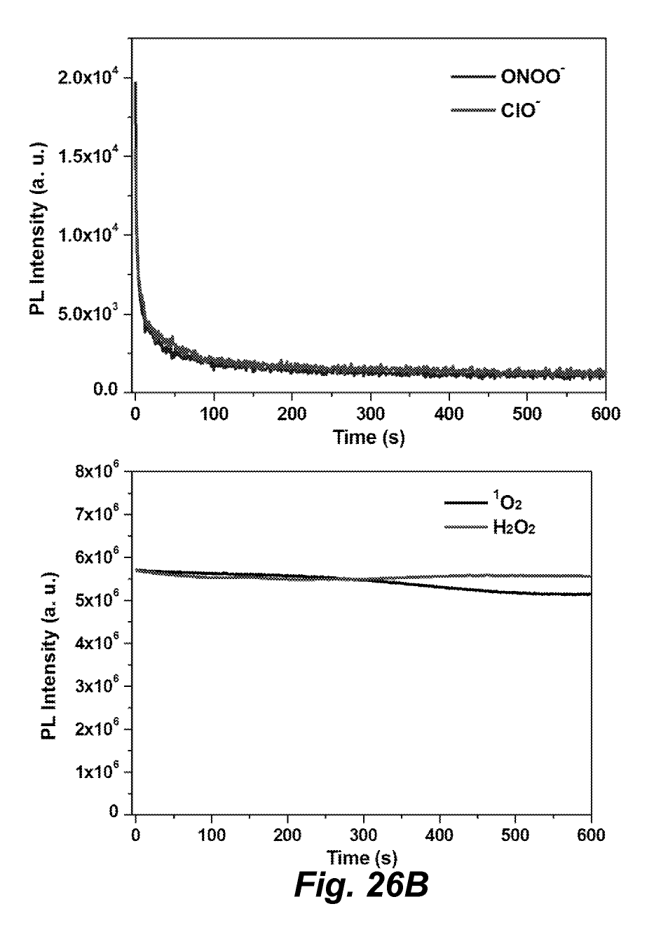

The elevated generation of ROS is a hallmark of many pathological processes, such as cancer, cardiomyopathy, stroke and bacterial infections (Medzhitov et al., (2008) Nature 454: 428-435; Szabo et al., (2007) Nat. Rev. Drug Discov. 6: 662-680). Therefore, imaging of ROS is critical to understanding both the etiology of these diseases and optimizing therapeutic interventions against these potentially life-threatening conditions. SPN1 itself has high stability toward ROS, as shown in FIGS. 25A-25C, and thus was coupled to a cyanine dye derivative (IR775S) that is sensitive to ROS-mediated oxidation, as shown in FIGS. 26A and 26B to design a ratiometric PA probe (RSPN) for ROS imaging (FIG. 17A).

### Example 6

Characterization of ROS-Synthesis of RSPNs:

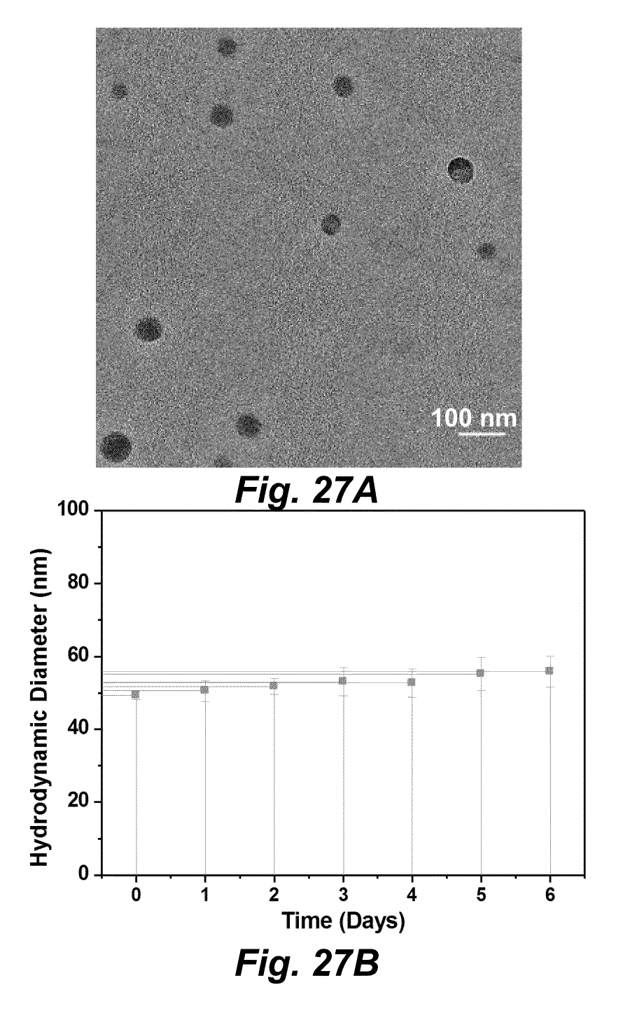

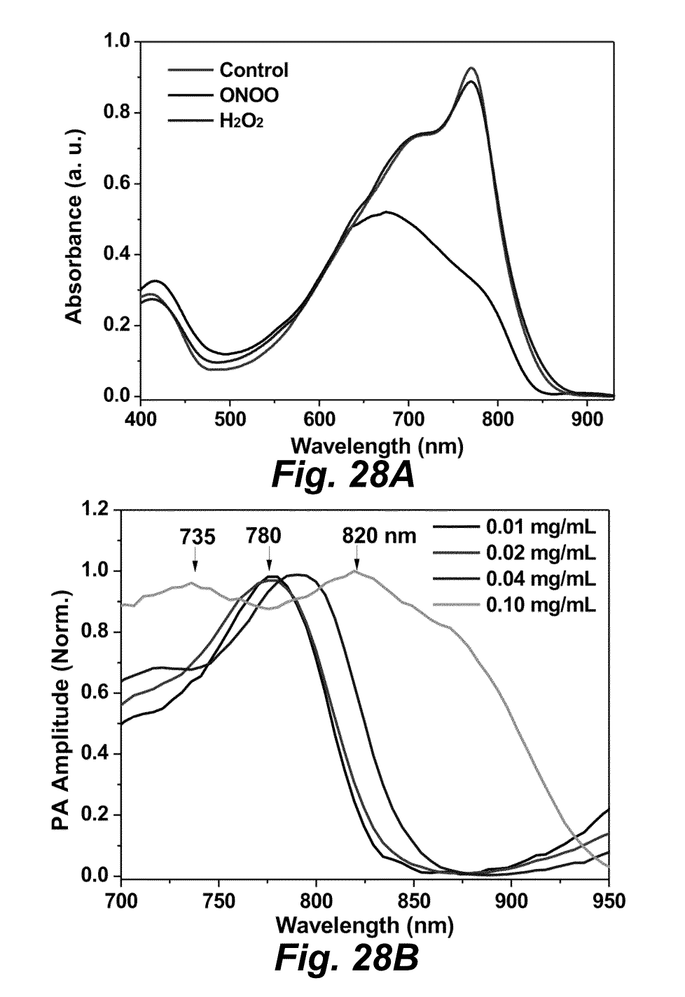

One-pot nanoprecipitation of 1R775S and PCPDTBT with 1,2-dipalmitoyl-sn-glycero-3-phosphocholine (DPPC) yielded the water-soluble RSPN (FIG. 17A), which possessed a small diameter (45 nm) and good size stability similar to SPN1 (FIGS. 27A and 27B). The average number of 1R775S per nanoparticle was estimated to be 50 based on the molar extinction coefficients of SPN1 and 1R775S. The PA spectrum of RSPN showed three maxima at 700, 735, and 820 nm (FIG. 17B) with nearly the same amplitude, which differed from its absorption spectrum (FIG. 28A). According to the PA spectrum of SPN1 (FIG. 11), the peak at 700 nm corresponded to the PCPDTBT in RSPN, while the peaks at 735 and 820 nm were observable for 1R775S at high dye concentrations (0.2 mM) (FIG. 28B). As the local dye concentration within the compact inner space of the nanoparticle is very high (corresponding to approximately 2.5 mM), the PA peaks at 735 and 820 stem from aggregated 1R775S in the RSPN core.

### Example 7

Photoacoustic Responses of RSPNs:

The PA responses of RSPN toward different ROS were examined in solution under physiological conditions. In the presence of peroxynitrite (ONOO⁻) and hypochlorite (ClO⁻), the PA peak of RSPN at 735 nm significantly decreased, and the peak at 820 nm almost disappeared, while the peak at 700 nm remained nearly the same (FIG. 17B). In contrast, in the presence of other ROS such as nitric oxide (*NO), hydroxyl radical (*OH), superoxide (O₂*⁻), singlet oxygen (¹O₂) and hydrogen peroxide (H₂O₂), the PA spectrum remained essentially unchanged (FIG. 4B). The ROS-dependent PA change at 820 nm, but not at 700 nm, was attributed to the ROS-mediated rapid oxidative decomposition of IR775S (FIGS. 26A and 26B) (Oushiki et al., (2010) J. Am. Chem. Soc. 132: 2795-2801), making RSPN amenable to dual-peak ratiometric detection of ROS (PA₇₀₀/PA₈₂₀).

The PA ratio of RSPN increased from 1.2 to 25 in the presence of ONOO⁻ and ClO⁻, but less than 4 for other ROS (FIG. 17C). The limit of detection for ONOO⁻ and ClO⁻ was determined to be approximately 50 nM. Thus, RSPN showed a large dynamic signal range and high sensitivity to detect both ROS at pathologically relevant concentrations (Szabo et al., (2007) Nat. Rev. Drug Discov. 6: 662-680).

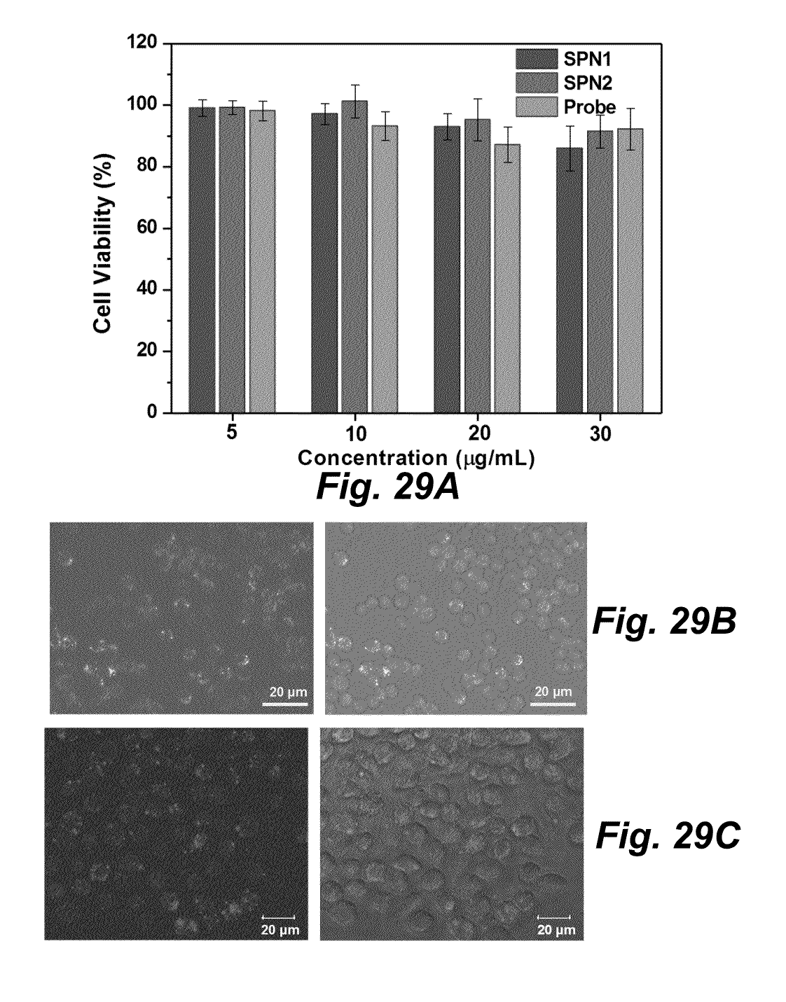

The capability of RSPN to detect endogenously generated ROS was then tested in cultured murine macrophage RAW264.7 cells, which are relevant to inflammation. After validating that SPNs have low cytotoxicity and efficient cell uptake (FIGS. 29A-29C), RSPN were incubated with RAW264.7, and cell pellets were loaded into an agar phantom for PA imaging. For cells in the resting state, a strong PA signal was observed at both 700 and 820 nm (FIG. 17D), giving rise to a PA₇₀₀/PA₈₂₀ of 1.4±0.43 (FIG. 17E).

To mimic the inflammatory condition that stimulates resting macrophages to produce ROS, such as ONOO⁻ and ClO⁻, RAW264.7 cells were pre-treated with a pathogen-associated molecular pattern (PAMP), bacterial cell wall lipopolysaccharide (LPS), and a dimerized soluble cytokine, interferon-γ (INF-γ). Following LPS/INF-γ stimulation, the PA signal at 820 nm was nearly abolished, (FIG. 17D), leading to an enhanced PA₇₀₀/PA₈₂₀ of 7.3±0.96 (FIG. 17E). When N-acetylcysteine (NAC), a free-radical scavenger with high membrane permeability (Winterbourn et al., (1999) Free Radical Bio. Med. 27: 322-328), was used to treat the cells along with LPS/INF-γ stimulation, PA₇₀₀/PA₈₂₀ returned to a low value (3.3±0.78). This result indicated that NAC scavenged endogenously generated ROS from macrophage cells and effectively inhibited the activation of RSPN.

An overlay of pseudocolored PA images (FIG. 17D) at 700 nm and 820 nm, facilitated the monitoring of ROS levels in cells. The dramatic ratiometric PA responses of RSPN were easily discernible between the resting RAW264.7 cells, the stimulated cells and the NAC-protected cells, with one color such as yellow representing a PA ratio near unity and another such as green representing a significant elevation of ROS.

RSPN was evaluated for the in vivo PA imaging of ROS in a murine model of acute edema induced by the intramuscular injection of zymosan into the thigh of living mice. Zymosan is a structural polysaccharide of the cell wall of Saccharomyces cerevisiae, which, similarly to LPS, can simulate the generation of ROS such as ONOO⁻ and ClO⁻ in vivo (Zhao et al., (2013) Mol. Pharmacol. 83: 167-178).

RSPN (3 μg) was administered intramuscularly into the same location of living mice 20 min after saline or zymosan treatment. The PA signal was simultaneously monitored at 700 and 820 nm, as illustrated by pseudo green and red colors, respectively. The PA amplitude at 700 nm for both control and zymosan-treated mice remained unchanged over time (FIG. 18A). The progressive enlargement in signalling area was attributed to the nanoparticle dispersion through tissue over time. In contrast, both the PA amplitude and signalling area at 820 nm for zymosan-treated mice significantly decreased over time (FIG. 18A), which was imperceptible for control mice. The superposition analysis clearly delineated a progressive pseudocolor variation from yellow to green for zymosan-treated mice but not for saline-treated mice (FIG. 18A), reporting the in situ generation of inflammatory ROS in zymosan-induced edema. Quantitative analysis revealed that PA₇₀₀/PA₈₂₀ gradually increased to 2.7±0.31 at 120 min post-injection for zymosan-treated mice, which was significantly elevated relative to the PA ratio for control mice (1.4±0.22) (FIG. 18B). Thus, RSPN effectively detected ROS produced in vivo using a dual-peak ratiometric PA contrast mechanism, demonstrating its potential for activatable PA imaging.

What is claimed:

1. A nanoparticle comprising an organic photovoltaic semiconductor polymer and a phospholipid, wherein the semiconductor polymer is near-infra red absorbing and generates a detectable photoacoustic signal and a fluorescent emission when irradiated by an incident activation energy.

2. The nanoparticle of claim 1, wherein the organic photovoltaic semiconductor polymer is selected from the group consisting of: poly(cyclopentadithiophene-alt-benzothiadiazole) (PCPDTBT), poly(acenaphthothienopyrazine-alt-benzodithiophene) (PATPBDT), poly[4,6-(dodecyl-thieno[3,4-b]thiophene-2-carboxylate)-alt-2,6-(4,8-dioctoxylbenzo[1,2-b:4,5-b]dit, poly[N-90-heptadecanyl-2,7carbazole-alt-3,6-bis(thiophen-5-yl)-2,5-dioctyl-2,5-dihydropyrrolo[3,4]pyrrole-1,4-dione], poly{4,8-bis[(2-ethylhexyl)oxy]benzo[1,2-b:4,5-b′]dithiophene-2,6-diyl-alt-3-fluoro-2-[(2-ethylhexyl], and poly[2,6(4,4′bis(ethylhexyl)dithieno[3,2-b:2′,3′-d]siloleyalt-(1,3-(5-octyl-4H-thieno[3,4-c]pyrrole]

3. The nanoparticle of claim 1, wherein the organic photovoltaic semiconductor polymer is selected from the group consisting of the polymers designated as SNP1, SNP2, SNP3, SNP4, and SNP5.

4. The nanoparticle of claim 1, wherein the phospholipid is selected from the group consisting of: 1,2-dipalmitoyl-sn-glycero-3-phosphocholine (DPPC), 1,2-dinonadecanoyl-sn-glycero-3-phosphocholine, 1,2-diarachidoyl-sn-glycero-3-phosphocholine, 1,2-dibehenoyl-sn-glycero-3-phosphocholine, 1,2-dilignoceroyl-sn-glycero-3-phosphocholine, 1,2-distearoyl-sn-glycero-3-phosphoethanolamine-N-[methoxy(polyethylene glycol)-2000] (ammonium salt), 1,2-dioleoyl-sn-glycero-3-phosphoethanolamine-N-[methoxy(polyethylene glycol)-2000] (ammonium salt), 1,2-dimyristoyl-sn-glycero-3-phosphoethanolamine-N-[methoxy(polyethylene glycol)-350] (ammonium salt), 1,2-distearoyl-sn-glycero-3-phosphoethanolamine-N-[methoxy(polyethylene glycol)-550] (ammonium salt), 1,2-distearoyl-sn-glycero-3-phosphoethanolamine-N-[methoxy(polyethylene glycol)-3000] (ammonium salt), and 1,2-distearoyl-sn-glycero-3-phosphoethanolamine-N-[methoxy(polyethylene glycol)-5000] (ammonium salt).

5. The nanoparticle of claim 1, wherein said nanoparticle further comprises a reactive oxygen species (ROS)-inactivated fluorophore.

6. The nanoparticle of claim 5, wherein the fluorophore is 2-[4′-(β-carboxyethylthio)-7′-(1″,3″,3″-trimethylindolenine)-3′,5′-trimethyleneheptatrien-1-yl]-1,3,3-trimethylindolenium perchlorate (IR775S).

7. The nanoparticle of claim 1, further comprising at least one therapeutic agent.

8. A pharmaceutically acceptable composition comprising a nanoparticle comprising an organic photovoltaic semiconductor polymer and a phospholipid, wherein the semiconductor polymer is characterized as near-infra red absorbing and generating a detectable photoacoustic signal and a fluorescent emission when irradiated by an incident activation energy.

9. The pharmaceutically acceptable composition of claim 8, wherein the semiconductor polymer is selected from the group consisting of: poly(cyclopentadithiophene-alt-benzothiadiazole) (PCPDTBT), poly(acenaphthothienopyrazine-alt-benzodithiophene) (PATPBDT), poly[4,6-(dodecyl-thieno[3,4-b]thiophene-2-carboxylate)-alt-2,6-(4,8-dioctoxylbenzo[1,2-b:4,5-b]dit, poly[N-90-heptadecanyl-2,7carbazole-alt-3,6-bis(thiophen-5-yl)-2,5-dioctyl-2,5-dihydropyrrolo[3,4]pyrrole-1,4-dione], poly{4,8-bis[(2-ethylhexyl)oxy]benzo[1,2-b:4,5-b′]dithiophene-2,6-diyl-alt-3-fluoro-2-[(2-ethylhexyl], and poly[2,6(4,4′bis(ethylhexyl)dithieno[3,2-b:2′,3′-d]siloleyalt-(1,3-(5-octyl-4H-thieno[3,4-c]pyrrole].

10. The pharmaceutically acceptable composition of claim 8, wherein the organic photovoltaic semiconductor polymer is selected from the group consisting of the polymers designated as SNP1, SNP2, SNP3, SNP4, and SNP5.

11. The pharmaceutically acceptable composition of claim 8, wherein the phospholipid is selected from the group consisting of 1,2-dipalmitoyl-sn-glycero-3-phosphocholine (DPPC), 1,2-dinonadecanoyl-sn-glycero-3-phosphocholine, 1,2-diarachidoyl-sn-glycero-3-phosphocholine, 1,2-dibehenoyl-sn-glycero-3-phosphocholine, 1,2-dilignoceroyl-sn-glycero-3-phosphocholine, 1,2-distearoyl-sn-glycero-3-phosphoethanolamine-N-[methoxy(polyethylene glycol)-2000] (ammonium salt), 1,2-dioleoyl-sn-glycero-3-phosphoethanolamine-N-[methoxy(polyethylene glycol)-2000] (ammonium salt), 1,2-dimyristoyl-sn-glycero-3-phosphoethanolamine-N-[methoxy(polyethylene glycol)-350] (ammonium salt), 1,2-distearoyl-sn-glycero-3-phosphoethanolamine-N-[methoxy(polyethylene glycol)-550] (ammonium salt), 1,2-distearoyl-sn-glycero-3-phosphoethanolamine-N-[methoxy(polyethylene glycol)-3000] (ammonium salt), and 1,2-distearoyl-sn-glycero-3-phosphoethanolamine-N-[methoxy(polyethylene glycol)-5000] (ammonium salt).

12. The pharmaceutically acceptable composition of claim 8, wherein said nanoparticle further comprises a reactive oxygen species (ROS)-inactivated fluorophore.

13. The pharmaceutically acceptable composition of claim 8, wherein the fluorophore is 2-[4′-(β-carboxyethylthio)-7′-(1″,3″,3″-trimethylindolenine)-3′,5′-trimethyleneheptatrien-1-yl]-1,3,3-trimethylindolenium perchlorate (IR775S).

14. The pharmaceutically acceptable composition of claim 8, further comprising a pharmaceutically acceptable carrier.

15. The pharmaceutically acceptable composition of claim 8, further comprising at least one therapeutic agent.

16. A method of molecular imaging, comprising the steps of:
(a) delivering to a human or non-human subject a pharmaceutically acceptable composition comprising a plurality of nanoparticles comprising an organic photovoltaic semiconductor polymer, and a phospholipid a reactive oxygen species (ROS)-inactivated fluorophore, wherein the semiconductor polymer is characterized as near-infra red absorbing and generating a first detectable photoacoustic signal spectrum when irradiated by an incident activation energy;
(b) irradiating the human or non-human subject with a first incident energy having a first wavelength and generating a first photoacoustic signal;
(c) irradiating the human or non-human subject with a second incident energy having a second wavelength and generating a second photoacoustic signal;
(d) determining the intensities of the first and the second photoacoustic signals;
(e) determining a first ratio of the intensities of the first and the second photoacoustic signals;
(f) comparing said first ratio with a second ratio determined from the nanoparticles before delivery to the human or non-human subject, whereby a difference in said first and second ratios indicates RONS degradation of the nanoparticles in the subject; and
(g) generating a ratiometric image indicating the difference in said first and second ratios relative to an image of the subject.

17. The method of claim 16, wherein the semiconductor polymer is selected from the group consisting of: poly(cyclopentadithiophene-alt-benzothiadiazole) (PCPDTBT), poly(acenaphthothienopyrazine-alt-benzodithiophene) (PATPBDT), poly[4,6-(dodecyl-thieno[3,4-b]thiophene-2-carboxylate)-alt-2,6-(4,8-dioctoxylbenzo[1,2-b:4,5-b]dit, poly[N-90-heptadecanyl-2,7carbazole-alt-3,6-bis(thiophen-5-yl)-2,5-dioctyl-2,5-dihydropyrrolo[3,4]pyrrole-1,4-dione], poly{4,8-bis[(2-ethylhexyl)oxy]benzo[1,2-b:4,5-b′]dithiophene-2,6-diyl-alt-3-fluoro-2-[(2-ethylhexyl], and poly[2,6(4,4′bis(ethylhexyl)dithieno[3,2-b:2′,3′-d]siloleyalt-(1,3-(5-octyl-4H-thieno[3,4-c]pyrrole]

18. The method of claim 16, wherein the organic photovoltaic semiconductor polymer is selected from the group consisting of the polymers designated as SNP1, SNP2, SNP3, SNP4, and SNP5.

19. The method of claim 18, wherein the phospholipid is selected from the group consisting of: 1,2-dipalmitoyl-sn-glycero-3-phosphocholine (DPPC), 1,2-dinonadecanoyl-sn-glycero-3-phosphocholine, 1,2-diarachidoyl-sn-glycero-3-phosphocholine, 1,2-dibehenoyl-sn-glycero-3-phosphocholine, 1,2-dilignoceroyl-sn-glycero-3-phosphocholine, 1,2-distearoyl-sn-glycero-3-phosphoethanolamine-N-[methoxy(polyethylene glycol)-2000] (ammonium salt), 1,2-dioleoyl-sn-glycero-3-phosphoethanolamine-N-[methoxy(polyethylene glycol)-2000] (ammonium salt), 1,2-dimyristoyl-sn-glycero-3-phosphoethanolamine-N-[methoxy(polyethylene glycol)-350] (ammonium salt), 1,2-distearoyl-sn-glycero-3-phosphoethanolamine-N-[methoxy(polyethylene glycol)-550] (ammonium salt), 1,2-distearoyl-sn-glycero-3-phosphoethanolamine-N-[methoxy(polyethylene glycol)-3000] (ammonium salt), and 1,2-distearoyl-sn-glycero-3-phosphoethanolamine-N-[methoxy(polyethylene glycol)-5000] (ammonium salt).

20. The method of claim 18, wherein the fluorophore is 2-[4′-(β-carboxyethylthio)-7′-(1″,3″,3″-trimethylindolenine)-3′,5-trimethyleneheptatrien-1-yl]-1,3,3-trimethylindolenium perchlorate (IR775S).
